SEQUENCE LISTING (50 sequences)

SEQ ID NO: 1 - DNA, 22 bp, Artificial Sequence
  Description of Artificial Sequence primer
gagatcctccgacaattcaacc

SEQ ID NO: 2 - DNA, 22 bp, Artificial Sequence
  Description of Artificial Sequence primer
aaacagcagctgatcctgactg

SEQ ID NO: 3 - DNA, 24 bp, Artificial Sequence
  Description of Artificial Sequence primer
aagagatccatggtgttcaaggac

SEQ ID NO: 4 - DNA, 24 bp, Artificial Sequence
  Description of Artificial Sequence primer
aggtaggcatactcattgtcatcg

SEQ ID NO: 5 - DNA, 24 bp, Artificial Sequence
  Description of Artificial Sequence primer
gtcagagggatcacaatctttcag

SEQ ID NO: 6 - DNA, 23 bp, Artificial Sequence
  Description of Artificial Sequence primer
gtcttgtcgttggacgttagttc

SEQ ID NO: 7 - DNA, 20 bp, Artificial Sequence
  Description of Artificial Sequence primer
ttgtgaaccaacacctgtgc

SEQ ID NO: 8 - DNA, 23 bp, Artificial Sequence
  Description of Artificial Sequence primer
gtgtgtagaagaagcctcgttcc

SEQ ID NO: 9 - DNA, 20 bp, Artificial Sequence
  Description of Artificial Sequence primer
atcttcgccctggagaagac

SEQ ID NO: 10 - DNA, 20 bp, Artificial Sequence
  Description of Artificial Sequence primer
cgtgcttcttcctccacttg

SEQ ID NO: 11 - DNA, 20 bp, Artificial Sequence
  Description of Artificial Sequence primer
gccatcatggaggatgacga

SEQ ID NO: 12 - DNA, 21 bp, Artificial Sequence
  Description of Artificial Sequence primer
tgccatccacttcacaggtag

SEQ ID NO: 13 - DNA, 20 bp, Artificial Sequence
  Description of Artificial Sequence primer
gccaacagtgtatgctcgaa

SEQ ID NO: 14 - DNA, 20 bp, Artificial Sequence
  Description of Artificial Sequence primer
tccgtgtccttaacccgtaa

SEQ ID NO: 15 - DNA, 20 bp, Artificial Sequence
  Description of Artificial Sequence primer
ggcatgactgaacctgcatc

SEQ ID NO: 16 - DNA, 21 bp, Artificial Sequence
  Description of Artificial Sequence primer
cgtatgaggtctggagtgcaa

SEQ ID NO: 17 - DNA, 20 bp, Artificial Sequence
  Description of Artificial Sequence primer
tccttgacctccagcaacga

SEQ ID NO: 18 - DNA, 20 bp, Artificial Sequence
  Description of Artificial Sequence primer
ggcctgtccatccttaagcc

SEQ ID NO: 19 - DNA, 20 bp, Artificial Sequence
  Description of Artificial Sequence primer
cccggattagtgcgtacgag

SEQ ID NO: 20 - DNA, 22 bp, Artificial Sequence
  Description of Artificial Sequence primer
gcaatggctttggtctaaccag

SEQ ID NO: 21 - DNA, 20 bp, Artificial Sequence
  Description of Artificial Sequence primer
actgtggcattgagacagac

SEQ ID NO: 22 - DNA, 20 bp, Artificial Sequence
  Description of Artificial Sequence primer
tttcggttaacccgggtaag

SEQ ID NO: 23 - DNA, 20 bp, Artificial Sequence
  Description of Artificial Sequence primer
aggtcaagtggtagccatga

SEQ ID NO: 24 - DNA, 21 bp, Artificial Sequence
  Description of Artificial Sequence primer
tgtactgaccaggagggatag

SEQ ID NO: 25 - DNA, 21 bp, Artificial Sequence
  Description of Artificial Sequence primer
cctcatgaagatcctcaccga

SEQ ID NO: 26 - DNA, 21 bp, Artificial Sequence
  Description of Artificial Sequence primer
ttgccaatggtgatgacctgg

SEQ ID NO: 27 - DNA, 22 bp, Artificial Sequence
  Description of Artificial Sequence primer
agaatatggtccctgccgtaga

SEQ ID NO: 28 - DNA, 22 bp, Artificial Sequence
  Description of Artificial Sequence primer
cgtctacttgctctgcatgctg

SEQ ID NO: 29 - DNA, 20 bp, Artificial Sequence
  Description of Artificial Sequence primer
tctttgggctctcctcgaca

SEQ ID NO: 30 - DNA, 21 bp, Artificial Sequence
  Description of Artificial Sequence primer
ggcagtactgcacgaacttcc

SEQ ID NO: 31 - DNA, 19 bp, Artificial Sequence
  Description of Artificial Sequence primer
acctgcttgatgccaacac

SEQ ID NO: 32 - DNA, 22 bp, Artificial Sequence
  Description of Artificial Sequence primer
gtcagattcaggaaccaggtag

SEQ ID NO: 33 - DNA, 19 bp, Artificial Sequence
  Description of Artificial Sequence primer
tttccagctagcagtactc

SEQ ID NO: 34 - DNA, 22 bp, Artificial Sequence
  Description of Artificial Sequence primer
tccctatgtgatgtaactagtc

SEQ ID NO: 35 - DNA, 22 bp, Artificial Sequence
  Description of Artificial Sequence primer
gtattcagaccgactgggtatc

SEQ ID NO: 36 - DNA, 22 bp, Artificial Sequence
  Description of Artificial Sequence primer
tagaatacatggcggaaaggtc

SEQ ID NO: 37 - DNA, 22 bp, Artificial Sequence
  Description of Artificial Sequence primer
gcacaaaggatggagacaacga

SEQ ID NO: 38 - DNA, 19 bp, Artificial Sequence
  Description of Artificial Sequence primer
ggttgtggccttgagcgaa

SEQ ID NO: 39 - DNA, 22 bp, Artificial Sequence
  Description of Artificial Sequence primer
tcatcaccttcctcctgtagtc

SEQ ID NO: 40 - DNA, 21 bp, Artificial Sequence
  Description of Artificial Sequence primer
aacctttgctctcctgtaggt

SEQ ID NO: 41 - DNA, 20 bp, Artificial Sequence
  Description of Artificial Sequence primer
aggcactttagacccatacc

SEQ ID NO: 42 - DNA, 21 bp, Artificial Sequence
  Description of Artificial Sequence primer
cctacctcaacaagaagtgtc

SEQ ID NO: 43 - DNA, 19 bp, Artificial Sequence
  Description of Artificial Sequence primer
cgagccctgatgatcgtag

SEQ ID NO: 44 - DNA, 20 bp, Artificial Sequence
  Description of Artificial Sequence primer
atgttggcaaacacagacac

SEQ ID NO: 45 - DNA, 22 bp, Artificial Sequence
  Description of Artificial Sequence primer
acaatgattcccaagggatcac

SEQ ID NO: 46 - DNA, 22 bp, Artificial Sequence
  Description of Artificial Sequence primer
cactaagctgggagatgcatac

SEQ ID NO: 47 - DNA, 19 bp, Artificial Sequence
  Description of Artificial Sequence primer
agagggcaaggagatcgac

SEQ ID NO: 48 - DNA, 19 bp, Artificial Sequence
  Description of Artificial Sequence primer
gatgtggatgcggatacgg

SEQ ID NO: 49 - DNA, 23 bp, Artificial Sequence
  Description of Artificial Sequence primer
caatccactaacaagcagtcaac

SEQ ID NO: 50 - DNA, 22 bp, Artificial Sequence
  Description of Artificial Sequence primer
caccacgcttaaatgacaactc

(19) United States
(12) Patent Application Publication
(10) Pub. No.: US 2021/0246429 A1
(43) Pub. Date: Aug. 12, 2021

(54) METHOD FOR PRODUCING PRIMITIVE GUT TUBE CELLS

(71) Applicants: KANEKA CORPORATION, Osaka-shi, Osaka (JP); NATIONAL CENTER FOR GLOBAL HEALTH AND MEDICINE, Tokyo (JP)

(72) Inventors: Masato IBUKI, Kobe-shi (JP); Hirotoshi MATSUTA, Kobe-shi (JP); Hitoshi OKOCHI, Tokyo (JP); Shigeharu YABE, Tokyo (JP); Satsuki FUKUDA, Tokyo (JP)

(73) Assignees: KANEKA CORPORATION, Osaka-shi, Osaka (JP); NATIONAL CENTER FOR GLOBAL HEALTH AND MEDICINE, Tokyo (JP)

(21) Appl. No.: 17/049,728

(22) PCT Filed: Apr. 26, 2019
(86) PCT No.: PCT/JP2019/017977

§ 371 Date: Oct. 22, 2020

(30) Foreign Application Priority Data
Apr. 27, 2018 (JP) 2018-087225
Dec. 28, 2018 (JP) 2018-247333

(51) Int. Cl. C12N 5/071; A61K 35/37

(52) U.S. Cl. CPC C12N 5/0679; C12N 2501/15; A61K 35/37

(57) ABSTRACT

A problem addressed by the present invention is to provide a method for producing primitive gut tube cells from endothermal cells that have been induced to differentiate from pluripotent stein cells, wherein the method allows the production of pancreatic β cells of high quality. The present invention provides a method for producing primitive gut tube (PGT) cells comprising a step of culturing, in the absence of a bone morphogenetic protein (BMP) signaling inhibitor, endodermal cells that have been induced to differentiate from pluripotent stem cells.

### TECHNICAL FIELD

The present invention relates to a method for producing primitive gut tube cells, and primitive gut tube cells.

### BACKGROUND ART

Considerable expectations are being placed on regenerative medicine as an alternative to organ transplantation, which has a donor shortage issue, and in the development of new therapies for intractable diseases and the like. Embryonic stem cells (ES cells) and induced pluripotent stem cells (iPS cells) have pluripotency and infinite proliferative capacity, and are thus expected to be able to serve as cell sources for preparing the cells required for regenerative medicine. In order to put regenerative medicine using such pluripotent stem cells into practice, a technique for efficiently inducing the differentiation of pluripotent stem cells into target somatic cells needs to be established, and various differentiation induction methods have been reported.

For example, pancreatic β cells are useful in cell therapy for diabetes. Thus, methods for efficiently producing pancreatic β cells from pluripotent stem cells have been explored. Non-Patent Document 1 is a review regarding processes for generating functional pancreatic β cells from human iPS cells. Non-Patent Document 2 describes a method for efficiently generating functional pancreatic β cells from human iPS cells. Non-Patent Document 2 describes that, in stage 1, iPS cells were differentiated into endodermal cells, and thereafter, in stage 2, the endodermal cells were induced to differentiate into primitive gut tube (PGT) cells by culturing the cells in a culture medium containing dorsomorphin, which is a bone morphogenetic protein (BMP) signaling inhibitor, SANT1, which is a Hedgehog (HH) signaling inhibitor, SB431542, which is a TGFβ signaling inhibitor, and FGF2.

### CITATION LIST

### Non-Patent Literature

[Non-Patent Document 1]
Larry Sai Weng Loo, MSc. et al., Diabetes Obes Metab., 2018:20-3-13
[Non-Patent Document 2]
Shigeharu G. Yabe et al., Journal of Diabetes, 2017 February, 9(2):168-179

### DISCLOSURE OF INVENTION

### Technical Problem

Although, as mentioned above, culturing methods for inducing the differentiation of pluripotent stem cells into pancreatic β cells have been reported, there is a need to improve the efficiency of differentiation induction to raise the quality of the cells as pancreatic β cells in view of the therapeutic effects as cell therapy formulations.

Accordingly, the present invention addresses the problem of providing a method for producing primitive gut tube cells from endodermal cells that have been induced to differentiate from pluripotent stem cells, wherein the method makes it possible to efficiently produce primitive gut tube cells from endodermal cells, and also providing a method as mentioned above that makes it possible to produce pancreatic β cells that are of high quality. Furthermore, the present invention addresses the problem of providing, as a cell therapy formulation, primitive gut tube cells that are able to differentiate into optimal pancreatic β cells.

### Solution to Problem

As a result of diligent investigations towards solving the abovementioned problem, the present inventors discovered that primitive gut tube cells can be produced by culturing, in the absence of a bone morphogenetic protein (BMP) signaling inhibitor, endodermal cells that have been induced to differentiate from pluripotent stem cells. Furthermore, the present inventors discovered that pancreatic β cells produced by induced differentiation from the obtained primitive gut tube cells exhibited excellent normalization activity of blood glucose level in diabetes model mice, and thus that the primitive gut tube cells according to the present invention are superior to conventional primitive gut tube cells in terms of being able to differentiate into pancreatic β cells that can provide high therapeutic effects. The present invention was completed on the basis of this discovery.

In other words, according to the present description, the following invention is provided.

<1> A method for producing primitive gut tube (PGT) cells comprising a step of culturing, in the absence of a bone morphogenetic protein (BMP) signaling inhibitor, endodermal cells that have been induced to differentiate from pluripotent stem cells.
<1-1> A method for producing primitive gut tube (PGT) cells comprising a step of culturing, in the absence of a bone morphogenetic protein (BMP) signaling inhibitor, endodermal cells that have been induced to differentiate from pluripotent stem cells, under culturing conditions suitable for inducing differentiation into primitive gut tube (PGT) cells.
<2> The method according to <1>, wherein the step of culturing, in the absence of a bone morphogenetic protein (BMP) signaling inhibitor, endodermal cells that have been induced to differentiate from pluripotent stem cells is performed in the absence of FGF2.
<3> The method according to <1> or <2>, wherein the step of culturing, in the absence of a bone morphogenetic protein (BMP) signaling inhibitor, endodermal cells that have been induced to differentiate from pluripotent stem cells is performed in the absence of a hedgehog (HH) signaling inhibitor.
<4> The method according to any one of <1> to <3>, wherein the step of culturing, in the absence of a bone morphogenetic protein (BMP) signaling inhibitor, endodermal cells that have been induced to differentiate from pluripotent stem cells is performed in the absence of a TGFβ signaling inhibitor.
<4A> The method according to any one of <1> to <4>, wherein the step of culturing, in the absence of a bone morphogenetic protein (BMP) signaling inhibitor, endodermal cells that have been induced to differentiate from pluripotent stem cells is performed in the presence of retinoic acid or an analog thereof.
<5> The method according to any one of <1> to <4>, wherein the step of culturing, in the absence of a bone morphogenetic protein (BMP) signaling inhibitor, endodermal cells that have been induced to differentiate from pluripotent stem cells is a step of culturing the endodermal cells in a culture medium containing insulin, transferrin, and selenous acid.
<6> The method according to any one of <1> to <5>, wherein the step of culturing, in the absence of a bone morphogenetic protein (BMP) signaling inhibitor, endodermal cells that have been induced to differentiate from pluripotent stem cells is a step of culturing the endodermal cells in a culture medium containing a B27 (registered trademark) supplement and/or FGF7.
<6A> The method according to any one of <1> to <6>, wherein the step of culturing, in the absence of a bone morphogenetic protein (BMP) signaling inhibitor, endodermal cells that have been induced to differentiate from pluripotent stem cells is a step of culturing the endodermal cells in a culture medium containing an FGF receptor signaling activator.
<6B> The method according to <6A>, wherein the FGF receptor signaling activator is FGF7.
<6C> The method according to any one of <1> to <6>, <6A> and <6B>, wherein the step of culturing, in the absence of a bone morphogenetic protein (BMP) signaling inhibitor, endodermal cells that have been induced to differentiate from pluripotent stem cells is a step of culturing the endodermal cells in a culture medium containing an insulin receptor signaling activator.
<6D> The method according to <6C>, wherein the insulin receptor signaling activator is insulin.
<7> The method according to any one of <1> to <6>, wherein the endodermal cells that have been induced to differentiate from pluripotent stem cells are endodermal cells that have been induced to differentiate by culturing a pluripotent stem cell population in a culture medium containing a TGFβ superfamily signaling activator, and thereafter culturing the cell population in a culture medium to which FGF2 and BMP4 are not added.
<7A> The method according to any one of <1> to <7>, wherein the endodermal cells that have been induced to differentiate from pluripotent stem cells are endodermal cells that have been induced to differentiate by the following steps (a) to (b):
(a) a step of suspension culturing pluripotent stem cells using a culture medium containing 2-mercaptoethanol to prepare a cell population; and
(b) a step of culturing the cell population in a culture medium containing a TGFβ superfamily signaling activator, and thereafter culturing the cell population in a culture medium to which FGF2 and BMP4 are not added.
<7B> The method according to <7A>, wherein the culture medium containing 2-mercaptoethanol is a culture medium to which activin A is not added.
<7C> The method according to either <7A> or <7B>, wherein the culture medium containing 2-mercaptoethanol is a culture medium to which a WNT signaling activator is not added.
<7D> The production method according to any one of <7A> to <7C>, wherein the culture medium containing 2-mercaptoethanol is a culture medium to which FGF2 is not added.
<7E> The production method according to any one of <7A> to <7D>, wherein the culture medium containing 2-mercaptoethanol is a culture medium to which TGFβ1 is not added.
<7F> The production method according to any one of <7A> to <7E>, wherein the culture medium containing 2-mercaptoethanol is a culture medium further containing insulin.
<7G> The production method according to any one of <7A> to <7F>, wherein the culture medium to which FGF2 and BMP4 are not added is a culture medium containing at least one or more substances selected from among insulin, transferrin, sodium selenite and ethanolamine.
<7H> The method according to any one of <7A> to <7G>, wherein the culture medium containing a TGFβ superfamily signaling activator and/or the culture medium to which FGF2 and BMP4 are not added is a culture medium further containing 2-mercaptoethanol.
<8> Primitive gut tube (PGT) cells wherein expression of at least one gene selected from the group consisting of the KIT gene, the RAP1A gene, the FGF11 gene, and the FGFR4 gene is elevated, and/or expression of at least one gene selected from the group consisting of the MDM2 gene, the CASP3 gene, and the CDK1 gene is reduced in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.
<9> The primitive gut tube (PGT) cells according to <8>, wherein expression of at least one gene selected from the group consisting of the IGFBP3 gene, the PTGDR gene, the LOX gene, the PAPPA gene, and the RAB31 gene is elevated in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.
<10> The primitive gut tube (PGT) cells according to <8> or <9>, wherein expression of at least one gene selected from the group consisting of the ANGPT2 gene, the CD47 gene, the CDC42EP3 gene, the CLDN18 gene, the CLIC5 gene, the PHLDA1 gene, and the SKAP2 gene is reduced in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.
<11> Primitive gut tube (PGT) cells wherein expression of at least one gene selected from the group consisting of the IGFBP3gene, the PTGDR gene, and the PAPPA gene is elevated, and/or expression of at least one gene selected from the group consisting of the ANGPT2 gene and the FRZB gene is reduced in comparison with endodermal cells that have been induced to differentiate from pluripotent stem cells.
<21> A cell population including primitive gut tube cells, wherein the cell population has the cell properties in (a) to (d) indicated below:
(a) in the cell population, the relative expression level of the FGF11 gene with respect to the expression level of the β-Actin gene is 0.01 or higher;
(b) in the cell population, the relative expression level of the FGFR4 gene with respect to the expression level of the β-Actin gene is 0.03 or higher;
(c) in the cell population, the relative expression level of the CASP3 gene with respect to the expression level of the β-Actin gene is 0.006 or lower; and
(d) in the cell population, the relative expression level of the CDK1 gene with respect to the expression level of the β-Actin gene is 0.02 or lower.
<22> The cell population according to <21>, wherein the relative expression level of the RAP1A gene with respect to the expression level of the β-Actin gene is 0.03 or higher.
<23> The cell population according to either <21> or <22>, wherein the relative expression level of the KIT gene with respect to the expression level of the β-Actin gene is 0.05 or higher.
<24> The cell population according to any one of <21> to <23>, wherein the relative expression level of the MDM2 gene with respect to the expression level of the β-Actin gene is 0.03 or lower.
<25> The cell population according to any one of <21> to <24>, wherein the relative expression level of the IGFBP3 gene with respect to the expression level of the OAZ1 gene is 10 or higher, the expression level of the PTGDR gene with respect to the expression level of the OAZ1 gene is 0.6 or higher, the relative expression level of the LOX gene with respect to the expression level of the OAZ1 gene is 0.6 or higher, the relative expression level of the PAPPA gene with respect to the expression level of the OAZ1 gene is 0.01 or higher, and the relative expression level of the RAB31 gene with respect to the expression level of the OAZ1 gene is 0.2 or higher.
<26> The cell population according to any one of <21> to <25>, wherein the relative expression level of the ANGPT2 gene with respect to the expression level of the OAZ1 gene is 0.0002 or lower, the relative expression level of the CD47 gene with respect to the expression level of the OAZ1 gene is 0.02 or lower, the relative expression level of the CDC42EP3 gene with respect to the expression level of the OAZ1 gene is 0.03 or lower, the relative expression level of the CLDN18 gene with respect to the expression level of the OAZ1 gene is 0.006 or lower, the relative expression level of the CLIC5 gene with respect to the expression level of the OAZ1 gene is 0.0001 or lower, the relative expression level of the PHLDA1 gene with respect to the expression level of the OAZ1 gene is 0.2 or lower, and the relative expression level of the SKAP2 gene with respect to the expression level of the OAZ1 gene is 0.01 or lower.
<27> Primitive gut tube (PGT) cells in which the expression of at least one or more genes selected from the group consisting of the IGFBP3 gene, the PTGDR gene, the LOX gene, the PAPPA gene, and the RAB31 gene is elevated and/or the expression of at least one or more genes selected from the group consisting of the ANGPT2 gene, the BMPR1B gene, the CD47 gene, the CDC42EP3 gene, the CLDN18 gene, the CLIC5 gene, the FRZB gene, the IGF2 gene, the PHLDA1 gene, and the SKAP2 gene is reduced in comparison with primitive gut tube (PGT) cells produced by culturing, in the presence of a bone morphogenetic protein (BMP) signaling inhibitor, a TGF-β signaling inhibitor, and a hedgehog (HH) signaling inhibitor, endodermal cells that have been induced to differentiate from pluripotent stem cells.

### Advantageous Effects of Invention

The production method of the present invention is able to efficiently produce primitive gut tube cells from endodermal cells and to obtain primitive gut tube cells that are able to differentiate into pancreatic β cells that exhibit excellent normalization activity of blood glucose level, and thus can provide a high-quality cell therapy formulation. Additionally, the pancreatic β cells derived from primitive gut tube cells produced by the present invention exhibit excellent normalization activity of blood glucose level and have excellent therapeutic effects as cell therapy formulations. Furthermore, the primitive gut tube cells produced by the present invention can differentiate into pancreatic β cells that are optimal for use as cell therapy formulations.

### BRIEF DESCRIPTION OF DRAWINGS

FIG. 1 indicates the results of analysis of the expression of primitive gut tube cell marker genes (HNF-1β, HNF-4α) in primitive gut tube cells induced to differentiate from human iPS cells.

FIG. 2 indicates the results of analysis of the expression of pancreatic β cell marker genes (INS, NKX6.1) in pancreatic β cells induced to differentiate from human iPS cells.

FIG. 3 indicates the results of measurement of the casual blood glucose level in a cell transplantation experiment in diabetes model mice.

FIG. 4 indicates the results of analysis of the expression of a pancreatic β cell marker gene (INS) in pancreatic β cells induced to differentiate from human iPS cells.

FIG. 5 indicates the results of quantitative RT-PCR on genes with elevated expression and genes with reduced expression in Example 1 in comparison to Comparative Example 5 and Reference Example 2.

FIG. 6 indicates the results of microarray analysis of genes in which the signal value in Example 1 was ten or more times that in Comparative Example 5.

FIG. 7 indicates the results of quantitative RT-PCR on genes with elevated expression in Example 1 in comparison to Comparative Example 5.

FIG. 8 indicates the results of microarray analysis of genes in which the signal value in Example 1 was one-tenth or less of that in Comparative Example 5.

FIG. 9 indicates the results of quantitative RT-PCR on genes with reduced expression in Example 1 in comparison to Comparative Example 5.

### BEST MODE FOR CARRYING OUT THE INVENTION

Hereinafter, embodiments of the present invention will be described in detail, but the following description is to facilitate understanding of the present invention. The scope of the present invention is not limited to the following embodiments, and other embodiments suitably substituted with features of the following embodiments by a person skilled in the art are also included in the scope of the present invention.

### Explanation of Terminology

In the present invention, “in the absence of an inhibitor” means “in a culture medium to which the inhibitor is not added”.

In connection with the culture medium in the present invention, the term “is not added” indicates that a factor such as a protein, a peptide, or a compound specified as not added to a culture or a conditioned medium is not exogenously added. If a factor such as a protein, a peptide, or a compound specified as not added to a culture or a conditioned medium is brought in by continuous culture operation, the amount of the factor is adjusted to be less than 1% (volume/volume), less than 0.5% (volume/volume), less than 0.1% (volume/volume), less than 0.05% (volume/volume), less than 0.01% (volume/volume), or less than 0.001% (volume/volume).

In connection with gene expression levels, the term “elevated” indicates that the expression of a gene is increased over that of a specific gene expression level in a cell population to be compared, and is, relative to the cell population to be compared, 1.1 times or more, 1.2 times or more, 1.3 times or more, 1.4 times or more, 1.5 times or more, 1.6 times or more, 1.7 times or more, 1.8 times or more, 1.9 times or more, 2.0 times or more, 2.1 times or more, 2.2 times or more, 2.3 times or more, 2.4 times or more, 2.5 times or more, 2.6 times or more, 2.7 times or more, 2.8 times or more, 2.9 times or more, 3.0 times or more, 3.1 times or more, 3.2 times or more, 3.3 times or more, 3.4 times or more, 3.5 times or more, 3.6 times or more, 3.7 times or more, 3.8 times or more, 3.9 times or more, 4.0 times or more, 4.1 times or more, 4.2 times or more, 4.3 times or more, 4.4 times or more, 4.5 times or more, 4.6 times or more, 4.7 times or more, 4.8 times or more, 4.9 times or more, 5.0 times or more, 5.1 times or more, 5.2 times or more, 5.3 times or more, 5.4 times or more, 5.5 times or more, 5.6 times or more, 5.7 times or more, 5.8 times or more, 5.9 times or more, 6.0 times or more, 6.1 times or more, 6.2 times or more, 6.3 times or more, 6.4 times or more, 6.5 times or more, 6.6 times or more, 6.7 times or more, 6.8 times or more, 6.9 times or more, 7.0 times or more, 7.1 times or more, 7.2 times or more, 7.3 times or more, 7.4 times or more, 7.5 times or more, 7.6 times or more, 7.7 times or more, 7.8 times or more, 7.9 times or more, 8.0 times or more, 8.1 times or more, 8.2 times or more, 8.3 times or more, 8.4 times or more, 8.5 times or more, 8.6 times or more, 8.7 times or more, 8.8 times or more, 8.9 times or more, 9.0 times or more, 9.1 times or more, 9.2 times or more, 9.3 times or more, 9.4 times or more, 9.5 times or more, 9.6 times or more, 9.7 times or more, 9.8 times or more, 9.9 times or more, 10 times or more, 11 times or more, 12 times or more, 13 times or more, 14 times or more, 15 times or more, 20 times or more, 30 times or more, 40 times or more, 50 times or more, 60 times or more, 70 times or more, 80 times or more, 90 times or more, 100 times or more, 250 times or more, 400 times or more, 450 times or more, 500 times or more, 750 times or more, 1000 times or more, 5000 times or more, or 10000 times or more.

In connection with gene expression levels, the term “reduced” indicates that the expression of a gene is reduced from that of a specific gene expression level in a cell population to be compared, and is, relative to the cell population to be compared, 1.1 times or less, 1.2 times or less, 1.3 times or less, 1.4 times or less, 1.5 times or less, 1.6 times or less, 1.7 times or less, 1.8 times or less, 1.9 times or less, 2.0 times or less, 2.1 times or less, 2.2 times or less, 2.3 times or less, 2.4 times or less, 2.5 times or less, 2.6 times or less, 2.7 times or less, 2.8 times or less, 2.9 times or less, 3.0 times or less, 3.1 times or less, 3.2 times or less, 3.3 times or less, 3.4 times or less, 3.5 times or less, 3.6 times or less, 3.7 times or less, 3.8 times or less, 3.9 times or less, 4.0 times or less, 4.1 times or less, 4.2 times or less, 4.3 times or less, 4.4 times or less, 4.5 times or less, 4.6 times or less, 4.7 times or less, 4.8 times or less, 4.9 times or less, 5.0 times or less, 5.1 times or less, 5.2 times or less, 5.3 times or less, 5.4 times or less, 5.5 times or less, 5.6 times or less, 5.7 times or less, 5.8 times or less, 5.9 times or less, 6.0 times or less, 6.1 times or less, 6.2 times or less, 6.3 times or less, 6.4 times or less, 6.5 times or less, 6.6 times or less, 6.7 times or less, 6.8 times or less, 6.9 times or less, 7.0 times or less, 7.1 times or less, 7.2 times or less, 7.3 times or less, 7.4 times or less, 7.5 times or less, 7.6 times or less, 7.7 times or less, 7.8 times or less, 7.9 times or less, 8.0 times or less, 8.1 times or less, 8.2 times or less, 8.3 times or less, 8.4 times or less, 8.5 times or less, 8.6 times or less, 8.7 times or less, 8.8 times or less, 8.9 times or less, 9.0 times or less, 9.1 times or less, 9.2 times or less, 9.3 times or less, 9.4 times or less, 9.5 times or less, 9.6 times or less, 9.7 times or less, 9.8 times or less, 9.9 times or less, 10 times or less, 20 times or less, 30 times or less, 40 times or less, 50 times or less, 60 times or less, 70 times or less, 80 times or less, 90 times or less, 100 times or less, 250 times or less, 400 times or less, 450 times or less, 500 times or less, 750 times or less, 1000 times or less, 5000 times or less, or 10000 times or less.

### <Aggregate>

The aggregate in the present invention may be referred to alternatively by the term “clump”, “cluster”, or “spheroid”, and generally refers to an assemblage of a group of cells that are not dissociated into single cells.

### <Pluripotent Stem Cell>

The pluripotent stem cells in the present invention refer to cells that have multilineage differentiation potential (pluripotency), being able to differentiate into all or multiple types of cells constituting a living body, and that can continue to proliferate endlessly while maintaining pluripotency in an in-vitro culture under suitable conditions. Specific examples thereof include embryonic stem cells (ES cells), pluripotent stem cells derived from embryonic primordial germ cells (EG cells; see Proc Natl Acad Sci USA, 1998, 95:13726-31), pluripotent stem cells derived from the spermary (GS cells; see Nature, 2008, 456:344-9), induced pluripotent stem cells (iPS cells), somatic stem cells (tissue stem cells), and the like. The pluripotent stem cells are preferably iPS cells or ES cells, and are more preferably iPS cells. The term “embryonic” refers to embryos derived by somatic nuclear transfer in addition to embryos derived by syngamy.

As the ES cells, it is possible to use cells derived from any warm-blooded animal, preferably a mammal. Examples of the mammals include mice, rats, guinea pigs, hamsters, rabbits, cats, dogs, sheep, swine, bovines, horses, goats, simians, and humans. Cells derived from humans are preferably used.

Specific examples of ES cells include ES cells of mammals or the like established by culturing early embryos before implantation, ES cells established by culturing early embryos produced by nuclear transfer of the nuclei of somatic cells, and ES cells obtained by modifying genes on chromosomes of these ES cells using genetic engineering techniques. The ES cells can be prepared in accordance with methods normally implemented in the relevant field and in publicly known documents. Mouse ES cells were established by Evans et al. (Evans et al., 1981, Nature 292:154-6) and Martin et al. (Martin, G. R. et al., 1981, Proc Natl Acad Sci 78: 7634-8) in 1981. Human ES cells were established by Thomson et al. (Thomson et al., Science, 1998, 282:1145-7) in 1998, and are available from WiCell Research Institute (website: http://www.wicell.org/, Madison, Wis., USA), the National Institute of Health in the USA, Kyoto University, or the like, or can be purchased, for example, from Cellartis (website: http://www.cellartis.com/, Sweden).

Induced pluripotent stem cells (iPS cells) are cells having pluripotency, obtained by reprogramming somatic cells. Multiple groups have succeeded in producing iPS cells, including such groups as the group including professor Shinya Yamanaka at Kyoto University, the group including Rudolf Jaenisch at the Massachusetts Institute of Technology, the group including James Thomson at the University of Wisconsin, and the group including Konrad Hochedlinger at Harvard University. For example, the international patent publication WO 2007/069666 describes a somatic nucleus reprogramming factor containing the gene products of an Oct family gene, a Klf family gene, and a Myc family gene, as well as a somatic nucleus reprogramming factor containing the gene products of an Oct family gene, a Klf family gene, a Sox family gene, and a Myc family gene. The publication further describes a method for producing induced pluripotent stem cells by reprogramming somatic nuclei, comprising a step of bringing the above-mentioned nucleus reprogramming factors into contact with somatic cells.

The types of somatic cells used for producing the iPS cells are not particularly limited, and any type of somatic cell may be used. Specifically, the somatic cells include all cells constituting a living body other than reproductive cells, and may be differentiated somatic cells or undifferentiated stem cells. The somatic cells may be from any of mammals, birds, fish, reptiles, and amphibians, and there are no particular limitations. However, they are preferably from mammals (for example, rodents such as mice, or primates such as humans), and are particularly preferably from mice or humans. When human somatic cells are used, somatic cells from fetuses, newborns, or adults may be used. Specific examples of somatic cells include fibroblasts (for example, dermal fibroblasts), epithelial cells (for example, gastric epithelial cells, liver epithelial cells, and alveolar epithelial cells), endothelial cells (for example, blood vessels and lymph vessels), nerve cells (for example, neurons and glial cells), pancreatic cells, white blood cells (B cells, T cells, etc.), marrow cells, muscle cells (for example, skeletal muscle cells, smooth muscle cells, and cardiac muscle cells), hepatic parenchymal cells, non-hepatic parenchymal cells, adipose cells, osteoblasts, cells constituting the periodontium (for example, periodontal membrane cells, cementoblasts, gingival fibroblasts, and osteoblasts) and cells constituting the kidneys, the eyes and the ears.

iPS cells are stem cells having the ability to self-replicate over a long period of time under prescribed culture conditions (for example, under the conditions for culturing ES cells) and having multipotency for differentiation into any of ectodermal cells, mesodermal cells, or endodermal cells, under prescribed differentiation induction conditions. Additionally, when the iPS cells are transplanted to test animals such as mice, they may be stem cells having the ability to form teratomas.

To produce iPS cells from somatic cells, at least one or more reprogramming genes are first introduced into the somatic cells. The reprogramming genes are genes encoding reprogramming factors having the function of reprogramming the somatic cells so as to become iPS cells. Specific examples of combinations of reprogramming genes include, but are not limited to, the following combinations:

(i) an Oct gene, a Klf gene, a Sox gene, and a Myc gene;
(ii) an Oct gene, a Sox gene, a NANOG gene, and a LIN28 gene;
(iii) an Oct gene, a Klf gene, a Sox gene, a Myc gene, a hTERT gene, and a SV40 large T gene; or
(iv) an Oct gene, a Klf gene, and a Sox gene.

Aside from the above, a method in which transgenes are further reduced (Nature, 2008 Jul. 31, 454(7204):646-50), a method using a low-molecular-weight compound (Cell Stem Cell, 2009 Jan. 9, 4(1):16-9; Cell Stem Cell, 2009 Nov. 6, 5(5):491-503), a method using transcription factor proteins instead of genes (Cell Stem Cell, 2009 May 8, 4(5):381-4), and the like have been reported, and the iPS cells may be iPS cells produced by any of these methods.

Although the mode of introduction of the reprogramming factors into cells is not particularly limited, examples include gene transfer using plasmids, transfection with synthetic RNA, and direct introduction of the proteins. Additionally, iPS cells produced by methods using microRNA or RNA, low-molecular-weight compounds, or the like may be used. The pluripotent stem cells, including ES cells and iPS cells, may be commercially available products or cells received by distribution, or may be newly produced.

As the iPS cells, it is possible to use, for example, the 253G1 cell line, the 253G4 cell line, the 201B6 cell line, the 201B7 cell line, the 409B2 cell line, the 454E2 cell line, the 606A1 cell line, the 610B1 cell line, the 648A1 cell line, the 1201C1 cell line, the 1205D1 cell line, 1210B2 cell line, the 1231A3 cell line, the 1383D2 cell line, the 1383D6 cell line, the iPS-TIG120-3f7 cell line, the iPS-TIG120-4f1 cell line, the iPS-TIG114-4f1 cell line, the RPChiPS771-2 cell line, the 15M63 cell line, the 15M66 cell line, the HiPS-RIKEN-1A cell line, the HiPS-RIKEN-2A cell line, the HiPS-RIKEN-12A cell line, the Nips-B2 cell line, the TkDN4-M cell line, the TkDA3-1 cell line, the TkDA3-2 cell line, the TkDA3-4 cell line, the TkDA3-5 cell line, the TkDA3-9 cell line, the TkDA3-20 cell line, the hiPSC 38-2 cell line, the MSC-iPSC1 cell line, the BJ-iPSC1 cell line, and the like.

As the ES cells, it is possible to use, for example, the KhES-1 cell line, the KhES-2 cell line, the KhES-3 cell line, the KhES-4 cell line, the KhES-5 cell line, the SEES1 cell line, the SEES2 cell line, the SEES3 cell line, the HUES8 cell line, the CyT49 cell line, the H1 cell line, the H9 cell line, the HS-181 cell line, and the like. Newly produced clinical-grade iPS cells or ES cells may also be used.

### <Signaling and Factors>

### (Bone Morphogenetic Protein (BMP) Signaling Inhibitor)

Bone morphogenetic protein (BMP) signals are signals that are mediated by bone morphogenetic protein (BMP) ligands, serving various roles in vertebrates. During embryogenesis, the dorsoventral axis is established by a BMP signaling gradient formed by the coordinated expression of ligands, receptors, coreceptors, and soluble antagonists. BMP is a regulator that is important for gastrulation, mesodermal induction, organogenesis, and cartilaginous bone formation, and that controls the fates of pluripotent stem cell populations.

BMP receptors comprise complexes of type I receptors (activin receptor-like kinase; ALK-1, ALK-2, ALK-3 or ALK-6) and type II receptors (ActRII, ActRIIB or BMPRII), and the activated type I receptor kinases cause phosphorylation of two serine residues located on the C terminus of the R-Smad (receptor-regulated Smad) protein. An R-Smad (Smad1, Smad5 or Smad8) that is phosphorylated by the ligand (BMP) binding to a receptor is called a BR-Smad (BMP R-Smad). Two molecules of R-Smad that have been phosphorylated form a heterotrimer with Smad4 and undergo nuclear translocation, thereby regulating the transcription of target genes.

The bone morphogenetic protein (BMP) signaling inhibitors are not particularly limited as long as they are substances that inhibit BMP signaling, which begins with ligands (BMP-4 or the like) binding to receptors. However, they are preferably substances that inhibit at least one of ALK-1, ALK-2, ALK-3, and ALK-6. Additionally, a substance that inhibits a ligand binding to a receptor (such as an antagonist antibody) may be used as the BMP signaling inhibitor.

The bone morphogenetic protein (BMP) signaling inhibitor is not particularly limited, but examples include dorsomorphin, LDN193189, LDN-214117, LDN-212854, K02288, ML347, and the like.

### (Hedgehog (HH) Signaling Inhibitor)

Hedgehog (HH) signals are known as being embryonic cell growth factors and morphogenetic factors. Additionally, they have been demonstrated as being capable of functioning to control tissue stem cells as well as homeostasis and tissue regeneration in adults. Abnormalities in embryonic HH signaling are a cause of congenital diseases such as holoprosencephaly, and the sustained activity of HH signaling in adults is considered to be associated with various forms of cancer including skin basal cell carcinoma and medulloblastoma. As hedgehog signaling ligands, three types of HH ligands (SHH, Sonic hedgehog; IHH, Indian hedgehog; and DHH, Desert hedgehog) are known in mammals. In the state in which there are no hedgehog ligands (off state), Patched, which is a receptor of the hedgehog family ligands, normally binds to Smoothened (Smo), which is a G protein-coupled transmembrane protein, and inhibits the association of Smoothened with the membrane. In the off state, SuFu and COS2 (which is Kif7 in vertebrates) isolate groups of Gli, which is a transcription factor that binds to microtubules, in the primary cilium. Gli is phosphorylated by PKA, CM and GSK-3, and Gli activating factors (Gli1 and Gli2 in mammals) are decomposed by β-TrCP, or Gli suppression factors (Gli3 or truncated Ci in Drosophila) are produced in a preserved pathway, which leads to suppression of the hedgehog target genes. In the activated state (on state), the hedgehog ligands bind to Patched, thereby allowing Smoothened, mediated by β-Arrestin, to move into the primary cilium, where the activity of G proteins associated therewith inhibits the inhibitory kinase activity that acts on Gli, allowing Gli to freely undergo nuclear translocation, thereby activating hedgehog target genes such as those for Cyclin D, Cyclin E, Myc, and Patched.

The hedgehog (HH) signaling inhibitor is not particularly limited as long as it is a substance that inhibits the above-mentioned hedgehog signaling, but examples thereof are substances that inhibit signaling by acting on Smo and the like. Additionally, antagonist antibodies that inhibit the binding of the hedgehog ligands to receptors such as Patched may also be used as the hedgehog signaling inhibitor.

The hedgehog (HH) signaling inhibitor is not particularly limited, but examples include SANT1, cyclopamine, sonidegib, PF-5274857, glasdegib, taladegib, BMS-833923, MK-4101, vismodegib, GANT61, jervine, HPI-4, and the like. For example, SANT-1 is an HH signaling antagonist that has strong cell penetrating properties and that inhibits signaling by binding directly to the Smo receptors, and thus can be favorably used.

### (TGFβ Signaling Inhibitor)

TGF-β receptor (TGFβ) signaling is signaling that involves ligands of transforming growth factor β (TGFβ), and that plays a central role in cell processes such as, for example, the growth, proliferation, differentiation and apoptosis of cells. The binding of TGFβ ligands to type II receptors (serine/threonine kinase), which gradually increases and phosphorylates type I receptors (ALK5), is involved in TGFβ signaling. Next, these type I receptors phosphorylate receptor-regulated SMADs (R-SMADs; for example, SMAD1, SMAD2, SMAD3, SMAD5, SMAD8, or SMAD9) that bind to SMAD4. Then, these SMAD complexes enter nuclei and serve roles in transcriptional regulation.

The TGFβ signaling inhibitor is not particularly limited as long as it is a substance that inhibits the above-mentioned TGFβ signaling, but examples thereof are substances that act on ALK5 and inhibit the phosphorylation thereof. Additionally, antagonist antibodies that inhibit the binding of TGFβ to receptors and the like may also be used as the TGFβ signaling inhibitor.

The TGFβ signaling inhibitor is not particularly limited, but examples include SB431542, galunisertib, LY2109761, SB525334, SB505124, GW788388, LY364947, RepSox, SD-208, vactosertib, LDN-212854, and the like.

### (Retinoic Acid)

Retinoic acid is a carboxylic acid derivative of vitamin A, and exists in the form of several stereoisomers such as all-trans retinoic acid (also known as tretinoin), 9-cis retinoic acid (also known as alitretinoin), and 13-cis retinoic acid (also known as isotretinoin). Retinoic acid serves a major role in the bioactivity of retinoids and carotenoids in the living body, as a natural ligand of retinoic acid receptor (RAR), which is one of nuclear receptors. RAR is known to form a heterodimer with retinoid X receptor (RXR, the ligand is 9-cis retinoic acid), and to serve as a ligand-inducible transcription factor that binds to promoters in specific target gene groups, thereby positively or negatively controlling, by the transcription level, the expression of the target gene groups. Even compounds having chemical structures that are not at all similar to vitamin A are referred to as retinoids, including synthetic compounds exhibiting extremely high binding affinity to these specific receptors.

### (Retinoic Acid Analog)

The retinoic acid analog is not particularly limited as long as it is a substance that, like retinoic acid, activates retinoic acid receptor (PAR), but examples include EC23, EC19, AC 261066, AC 55649, adapalene, AM 580, AM 80, BMS 753, BMS 961, CD 1530, C2314, CD 437, Ch 55, isotretinoin, tazarotene, TTNPB, and the like.

### (Insulin Receptor Signaling Activator)

Insulin receptors are expressed in the liver, skeletal muscles, adipose tissue, nerve cells, and the like, and insulin receptor signaling is known to be involved in the formation, maintenance and repair of the neural network. Insulin is an important hormone that regulates important energy functions such as glucose and lipid metabolism. Insulin activates insulin receptor (IR) tyrosine kinase and performs recruitment and phosphorylation of different substrate adapters such as the IRS (insulin receptor substrate) family. Tyrosine-phosphorylated IRS provides binding sites to many signaling partners. Among these, PI3K (phosphoinositide 3-kinase) plays an important role in insulin function, mainly through the activation of Akt (protein kinase B) and PKC (protein kinase C). Activated Akt causes glycogen synthesis by inhibiting GSK-3 (glycogen synthase kinase), protein synthesis by means of mTOR (mammalian target of rapa) and downstream factors, and cell survival by inhibiting proapoptotic factors (Bad, transcription factor Forkhead family, GSK-3, etc.). Insulin receptor signaling also has cell growth and cell division effects, and as with the activation of the Ras/MAPK pathway, Akt cascades are mainly involved in the effects thereof.

Although the insulin receptor signaling activator is not particularly limited as long as it is a substance that activates the above-mentioned insulin receptor signaling, examples include ligands that bind to insulin receptor and IGF receptor. Additionally, it may be a substance that directly or indirectly activates PI3K, PKC or Akt.

The insulin receptor signaling activator is preferably insulin, insulin-like growth factor-1 (IGF-1), IGF-2, or the like. Additionally, PI3-kinase activator (Santa Cruz, product number sc-3036), 740 Y-P and the like, which are PI3K activators, can also be used as insulin receptor signaling activators.

### (FGF Receptor Signaling Activator)

FGF (fibroblast growth factor) receptor signaling is signaling that is mediated by FGF receptors and that occurs on the RAS-MAPK pathway and the PI3K-AKT pathway. It is involved in various cell functions such as cell proliferation, cell death, angiogenesis, epithelial-to-mesenchymal transitions (EMT), and the like, and also serves an important role in controlling embryogenesis and post-natal development of the skeletal structure.

It is sufficient for the FGF receptor signaling activator to be a substance that activates signaling as mentioned above, and typical examples thereof are ligands (FGF family) that bind to FGF receptors. Additionally, activators of the RAS-MAPK pathway and the PI3K-AKT pathway may also be used as FGF receptor signaling activators.

Examples of FGF receptor signaling activators include the FGF family, among which FGF7, FGF3, FGF10, FGF22, FGF1, FGF2, FGF4, FGF5, FGF6, FGF8, FGF17, FGF18, FGF9, FGF16, FGF20, FGF19, FGF21, FGF23, and the like are preferable, and FGF7 is particularly preferable.

### (TGFβ Superfamily Signaling Activator)

TGFβ superfamily signaling plays a very important role in the regulation of cell proliferation, differentiation, and the development of a wide variety of biological systems. In general, signaling is initiated by serine/threonine receptor kinase multimer formation caused by ligands, and by the phosphorylation of intracellular signaling molecules such as Smad1/5/8 for the bone morphogenetic protein (BMP) pathway, or by the phosphorylation of Smad2/3 for the TGFβ/activin pathway and the NODAL/activin pathway. The phosphorylation of the carboxyl group terminals of Smads by activated receptors results in the formation of partners with Smad4, which is a signal transducer similar thereto, promoting nuclear translocation. It is known that activated Smads control various biological effects by partnering with transcription factors to perform transcriptional regulation that is specific to the cell state.

Examples of genes involved in the TGFβ superfamily signaling pathway include the activin A gene, the BMP2 gene, the BMP3 gene, the BMP4 gene, the BMP5 gene, the BMP6 gene, the BMP7 gene, the BMP8 gene, the BMP13 gene, the GDF2 (growth differentiation factor 2) gene, the GDF3 gene, the GDF5 gene, the GDF6 gene, the GDF7 gene, the GDF8 gene, the GDF11 gene, the TGF-01 gene, the TGF-02 gene, the TGF-03 gene, the AMH (anti-Mullerian hormone) gene, the paired-like homeodomain 2 (PITX2) gene, the NODAL gene, and the like.

The TGFβ superfamily signaling activator is not particularly limited as long as it is a substance that activates signaling on the bone morphogenetic protein (BMP) pathway, the TGFβ/activin pathway, and/or the NODAL/activin pathway. For example, it is possible to use activin A, BMP2, BMP3, BMP4, BMP5, BMP6, BMP7, BMP8, BMP13, GDF2, GDF5, GDF6, GDF7, GDF8, GDF11, TGF-01, TGF-02, TGF-03, AMH, PITX2, and/or NODAL. In particular, a substance that activates signaling on the TGFβ/activin pathway can be favorably used. Specifically, it is preferable to use at least one type selected from the group consisting of activin A and BMP4, and it is particularly preferable to use both activin A and BMP4.

### (WNT Signaling Activator)

WNT signaling refers to a series of actions to promote nuclear translocation of β-catenin, and to activate the functions thereof as a transcription factor. WNT signaling is caused by intercellular interactions, and for example, includes a series of processes in which a protein known as WNT3A, secreted from a certain cell, acts on another cell, causing β-catenin in the cell to undergo nuclear translocation and to act as a transcription factor. The series of processes triggers the first phenomena of organ construction, such as epithelial-mesenchymal interactions. WNT signaling is known to control various cell functions including proliferation and differentiation of cells, and cell motility in organogenesis and early development, by the activation of three pathways, namely, the β-catenin pathway, the PCP pathway, and the Ca²⁺ pathway.

Examples of genes involved in the WNT signaling pathway include the WNT3A gene and the like.

The WNT signaling activator is not particularly limited, and may be of any type as long as it exhibits inhibitory activity against glycogen synthase kinase-3 (GSK-3). It is possible to use, for example, a bis-indolo (indirubin) compound (BIO) ((2′Z,3′E)-6-bromoindirubin-3′-oxime), an acetoxime analog thereof, namely, BIO-acetoxime (2′Z,3′E)-6-bromoindirubin-3′-acetoxime), a thiadiazolidine (TDZD) analog (4-benzyl-2-methyl-1,2,4-thiadiazolidine-3,5-dione), an oxothiadiazolidine-3-thione analog (2,4-dibenzyl-5-oxothiadiazolidine-3-thione), a thienyl α-chloromethyl ketone compound (2-chloro-1-(4,4-dibromo-thiophen-2-yl)-ethanone), a phenyl α-bromomethyl ketone compound (α-4-dibromoacetophenone), a thiazole-containing urea compound (N-(4-methoxybenzyl)-N′-(5-nitro-1,3-thiazol-2-yl)urea), a GSK-3β peptide inhibitor such as H-KEAPPAPPQSpP-NH₂, particularly preferably CHIR99021 (CAS: 252917-06-9), or the like. WNT3A can also be favorably used.

### [1] Method for Producing Primitive Gut Tube (PGT) Cells

The method for producing primitive gut tube (PGT) cells according to the present invention is a method including a step of culturing, in the absence of a bone morphogenetic protein (BMP) signaling inhibitor, endodermal cells that have been induced to differentiate from pluripotent stem cells, under culture conditions that are suitable for inducing differentiation into primitive gut tube (PGT) cells. The induction of differentiation from pluripotent stem cells to endodermal cells will be described below.

The culture conditions suitable for inducing differentiation into primitive gut tube (PGT) cells are not particularly limited as long as they are culture conditions that can favorably induce differentiation, into primitive gut tube (PGT) cells, of endodermal cells that have been induced to differentiate from pluripotent stem cells.

The differentiation induction medium is not particularly limited as long as it is a culture medium that can induce the differentiation of endodermal cells into primitive gut tube (PGT) cells. As one embodiment, the cells may be cultured in the differentiation-inducing culture media indicated below.

In accordance with the types of cells that are used, an MEM medium, a BME medium, a DMEM medium, a DMEM/F12 medium, an αMEM medium, an IMDM medium, an ES medium, a DM-160 medium, a Fisher medium, an F12 medium, a WE medium, an RPMI1640 medium, an Essential 6™ medium (Thermo Fisher Scientific), or the like may be used. Additionally, a culture medium obtained by mixing two or more culture media arbitrarily selected from the aforementioned culture media may be used as needed. There are no particular limitations as long as it is a culture medium that can be used as a culture medium for animal cells.

The differentiation induction medium may further contain bovine serum albumin (BSA) or human serum albumin (HSA). Preferably, the BSA or HSA contains 2 mg/g or less of lipids and 0.2 mg/g or less of free fatty acids.

The lower limit of the amount of BSA added to the culture medium is preferably 0.01% (% by weight), more preferably 0.05%, more preferably 0.10%, more preferably 0.15%, more preferably 0.20%, and more preferably 0.25%. The upper limit of the amount of BSA added to the culture medium is preferably 1.00%, more preferably 0.90%, more preferably 0.80%, more preferably 0.70%, more preferably 0.60%, more preferably 0.50%, more preferably 0.40%, more preferably 0.30%, and more preferably 0.25%.

The differentiation induction medium may further contain sodium pyruvate.

The lower limit of the amount of sodium pyruvate added to the culture medium is preferably 0.01 mmol/L, more preferably 0.05 mmol/L, more preferably 0.1 mmol/L, more preferably 0.2 mmol/L, more preferably 0.5 mmol/L, more preferably 0.6 mmol/L, more preferably 0.7 mmol/L, more preferably 0.8 mmol/L, more preferably 0.9 mmol/L, and more preferably 1 mmol/L. The upper limit of the amount of sodium pyruvate added to the culture medium is preferably 20 mmol/L, more preferably 15 mmol/L, more preferably 10 mmol/L, more preferably 5 mmol/L, more preferably 4 mmol/L, more preferably 3 mmol/L, more preferably 2 mmol/L, and more preferably 1 mmol/L.

The differentiation induction medium may further contain NEAA (for example, 1× non-essential amino acids (NEAA, Wako) or the like).

The lower limit of the amount of NEAA contained in the culture medium is preferably 0.05×NEAA, more preferably 0.1×NEAA, more preferably 0.5×NEAA, more preferably 0.6×NEAA, more preferably 0.7×NEAA, more preferably 0.8×NEAA, more preferably 0.9×NEAA, and more preferably 1×NEAA. The upper limit of the amount of NEAA contained in the culture medium is preferably 20×NEAA, more preferably 15×NEAA, more preferably 10×NEAA, more preferably 5×NEAA, more preferably 4×NEAA, more preferably 3×NEAA, more preferably 2×NEAA, and more preferably 1×NEAA.

The differentiation induction medium may further contain antibiotics such as penicillin and streptomycin.

The lower limit of the amount of penicillin contained in the culture medium is preferably 1 unit/mL, more preferably 5 units/mL, more preferably 10 units/mL, more preferably 20 units/mL, more preferably 30 units/mL, more preferably 40 units/mL, more preferably 50 units/mL, more preferably 60 units/mL, more preferably 70 units/mL, more preferably 80 units/mL, more preferably 90 units/mL, and more preferably 100 units/mL. The upper limit is preferably 1000 units/mL, more preferably 500 units/mL, more preferably 400 units/mL, more preferably 300 units/mL, more preferably 200 units/mL, and more preferably 100 units/mL.

Additionally, the lower limit of the amount of streptomycin contained in the culture medium is preferably 10 μg/mL, more preferably 20 μg/mL, more preferably 30 μg/mL, more preferably 40 μg/mL, more preferably 50 μg/mL, more preferably 60 μg/mL, more preferably 70 μg/mL, more preferably 80 μg/mL, more preferably 90 μg/mL, and more preferably 100 μg/mL. The upper limit is preferably 1000 μg/mL, more preferably 500 μg/mL, more preferably 400 μg/mL, more preferably 300 μg/mL, more preferably 200 μg/mL, and more preferably 100 μg/mL.

### [2] Differentiation-Inducing Factor Used to Induce Differentiation to Primitive Gut Tube (PGT) Cells, and Other Additives

In the method for producing primitive gut tube (PGT) cells according to the present invention, other differentiation-inducing factors and other additives is not particularly limited as long as the method is performed in the absence of a bone morphogenetic protein (BMP) signaling inhibitor under culture conditions that are suitable for inducing the differentiation, into primitive gut tube (PGT) cells, of endodermal cells that have been induced to differentiate from pluripotent stem cells. However, in order to improve the differentiation induction efficiency and to produce primitive gut tube (PGT) cells that can differentiate into superior pancreatic β cells, the step of culturing, in the absence of a bone morphogenetic protein (BMP) signaling inhibitor, endodermal cells that have been induced to differentiate from pluripotent stem cells is preferably performed in the absence of FGF2.

Additionally, the step of culturing, in the absence of a bone morphogenetic protein (BMP) signaling inhibitor, endodermal cells that have been induced to differentiate from pluripotent stem cells, under culture conditions that are suitable for inducing the differentiation, into primitive gut tube (PGT) cells, of the endodermal cells that have been induced to differentiate from pluripotent stem cells is also preferably performed in the absence of a hedgehog (HH) signaling inhibitor. By culturing the aforementioned endodermal cells in the absence of a hedgehog (HH) signaling inhibitor, it is possible to improve the differentiation induction efficiency and to produce primitive gut tube (PGT) cells that can differentiate into superior pancreatic β cells.

Furthermore, the step of culturing, in the absence of a bone morphogenetic protein (BMP) signaling inhibitor, endodermal cells that have been induced to differentiate from pluripotent stem cells is also preferably performed in the absence of a TGFβ signaling inhibitor. By culturing the aforementioned endodermal cells in the absence of a TGFβ signaling inhibitor, it is possible to improve the differentiation induction efficiency and to produce primitive gut tube (PGT) cells that can differentiate into superior pancreatic β cells.

Furthermore, the step of culturing, in the absence of a bone morphogenetic protein (BMP) signaling inhibitor, endodermal cells that have been induced to differentiate from pluripotent stem cells is also preferably performed in the presence of retinoic acid or an analog thereof. By culturing the aforementioned endodermal cells in the presence of retinoic acid or an analog thereof, it is possible to improve the differentiation induction efficiency and to produce primitive gut tube (PGT) cells that can differentiate into superior pancreatic β cells.

Meanwhile, in order to improve the differentiation induction efficiency and to produce primitive gut tube (PGT) cells that can differentiate into superior pancreatic β cells, the step of culturing, in the absence of a bone morphogenetic protein (BMP) signaling inhibitor, endodermal cells induced from pluripotent stem cells is preferably a step of culturing the endodermal cells in a culture medium containing an insulin receptor signaling activator.

The lower limit of the amount of the insulin receptor signaling activator added to the differentiation induction medium is preferably 0.001 mg/L, more preferably 0.01 mg/L, more preferably 0.1 mg/L, more preferably 1 mg/L, more preferably 2 mg/L, and more preferably 3 mg/L. The upper limit of the amount of the insulin receptor signaling activator added to the culture medium is preferably 1000 mg/L, more preferably 500 mg/L, more preferably 100 mg/L, more preferably 90 mg/L, more preferably 80 mg/L, more preferably 70 mg/L, more preferably 60 mg/L, more preferably 50 mg/L, more preferably 40 mg/L, more preferably 30 mg/L, more preferably 20 mg/L, and more preferably 10 mg/L.

Additionally, the step of culturing the endodermal cells induced to differentiate from pluripotent stem cells, in the absence of a bone morphogenetic protein (BMP) signaling inhibitor, preferably involves culturing the endodermal cells in a culture medium containing insulin, transferrin, and selenous acid.

The insulin, transferrin, and selenous acid may be contained in the culture medium in the form of a commercially available mixture such as a B27 supplement. Additionally, ethanolamine may be contained in addition to insulin, transferrin, and selenous acid.

The lower limit of the amount of transferrin added to the culture medium is preferably 0.001 mg/L, more preferably 0.01 mg/L, more preferably 0.1 mg/L, more preferably 1 mg/L, more preferably 1.1 mg/L, more preferably 1.2 mg/L, more preferably 1.3 mg/L, more preferably 1.4 mg/L, more preferably 1.5 mg/L, more preferably 1.6 mg/L, and more preferably 1.65 mg/L. The upper limit of the amount of transferrin added to the culture medium is preferably 1000 mg/L, more preferably 500 mg/L, more preferably 100 mg/L, more preferably 90 mg/L, more preferably 80 mg/L, more preferably 70 mg/L, more preferably 60 mg/L, more preferably 50 mg/L, more preferably 40 mg/L, more preferably 30 mg/L, more preferably 20 mg/L, more preferably 10 mg/L, more preferably 9 mg/L, more preferably 8 mg/L, more preferably 7 mg/L, more preferably 6 mg/L, more preferably 5 mg/L, more preferably 4 mg/L, more preferably 3 mg/L, and more preferably 2 mg/L.

The lower limit of the amount of selenous acid added to the culture medium is preferably 0.001 μg/L, more preferably 0.01 μg/L, more preferably 0.1 μg/L, more preferably 1 μg/L, more preferably 1.1 μg/L, more preferably 1.2 μg/L, more preferably 1.3 μg/L, more preferably 1.4 μg/L, more preferably 1.5 μg/L, more preferably 1.6 μg/L, more preferably 1.7 μg/L, more preferably 1.8 μg/L, more preferably 1.9 μg/L, and more preferably 2 μg/L. The upper limit of the amount of selenous acid added to the culture medium is preferably 1000 μg/L, more preferably 500 μg/L, more preferably 100 μg/L, more preferably 90 μg/L, more preferably 80 μg/L, more preferably 70 μg/L, more preferably 60 μg/L, more preferably 50 μg/L, more preferably 40 μg/L, more preferably 30 μg/L, more preferably 20 μg/L, more preferably 10 μg/L, more preferably 9 m/L, more preferably 8 μg/L, and more preferably 7 μg/L.

In the step of culturing the endodermal cells induced to differentiate from pluripotent stem cells, in the absence of a bone morphogenetic protein (BMP) signaling inhibitor, the endodermal cells are preferably cultured in a culture medium containing an FGF receptor signaling activator, among which the endodermal cells are preferably cultured in a differentiation induction medium containing FGF7. However, as mentioned above in the present description, the endodermal cells are preferably cultured in the absence of FGF2 in order to more effectively induce differentiation and to produce primitive gut tube (PGT) cells that are able to induce differentiation into superior pancreatic β cells.

The lower limit of the amount of the FGF receptor signaling activator added to the culture medium is preferably 1 ng/mL, more preferably 5 ng/mL, more preferably 10 ng/mL, more preferably 20 ng/mL, more preferably 30 ng/mL, more preferably 40 ng/mL, and more preferably 50 ng/mL. The upper limit of the amount of the FGF receptor signaling activator added to the culture medium is preferably 500 ng/mL, more preferably 400 ng/mL, more preferably 300 ng/mL, more preferably 200 ng/mL, more preferably 100 ng/mL, more preferably 90 ng/mL, more preferably 80 ng/mL, more preferably 70 ng/mL, more preferably 60 ng/mL, and more preferably 50 ng/mL.

Preferably, the step of culturing the endodermal cells induced to differentiate from pluripotent stem cells, in the absence of a bone morphogenetic protein (BMP) signaling inhibitor, is a step of culturing the endodermal cells in a culture medium containing a B27 (registered trademark) supplement and/or FGF7.

The lower limit of the amount of the B27 (registered trademark) supplement added to the culture medium is preferably 0.01%, more preferably 0.1%, more preferably 0.2%, more preferably 0.3%, more preferably 0.4%, more preferably 0.5%, more preferably 0.6%, more preferably 0.7%, more preferably 0.8%, and more preferably 0.9%. The upper limit of the amount of the B27 (registered trademark) supplement added to the culture medium is preferably 10%, more preferably 9%, more preferably 8%, more preferably 7%, more preferably 6%, more preferably 5%, more preferably 4%, more preferably 3%, more preferably 2%, and more preferably 1%.

The lower limit of the amount of FGF7 added to the culture medium is preferably 1 ng/mL, more preferably 5 ng/mL, more preferably 10 ng/mL, more preferably 20 ng/mL, more preferably 30 ng/mL, more preferably 40 ng/mL, and more preferably 50 ng/mL. The upper limit of the amount of FGF7 added to the culture medium is preferably 500 ng/mL, more preferably 400 ng/mL, more preferably 300 ng/mL, more preferably 200 ng/mL, more preferably 100 ng/mL, more preferably 90 ng/mL, more preferably 80 ng/mL, more preferably 70 ng/mL, more preferably 60 ng/mL, and more preferably 50 ng/mL.

Additionally, the differentiation induction medium may contain a serum component or a serum replacement component aside from those mentioned above. Examples of the serum component or the serum replacement component include albumin, fatty acids, collagen precursors, trace elements (for example, zinc or selenium), N2 Supplement, N21 Supplement (R&D Systems), NeuroBrew-21 supplement (Miltenyi Biotec), KnockOut serum replacement (KSR), 2-mercaptoethanol, 3′thiolglycerol, and equivalents thereof.

Various additives, antibiotics, antioxidants, and the like may be further added to the differentiation induction medium. For example, it is possible to add 0.1 mM to 5 mM of sodium pyruvate, 0.1% to 2% (volume/volume) of non-essential amino acids, 0.1% to 2% (volume/volume) of penicillin, 0.1% to 2% (volume/volume) of streptomycin, and 0.1% to 2% (volume/volume) of amphotericin B, catalase, glutathione, galactose, retinoic acid (vitamin A), superoxide dismutase, ascorbic acid (vitamin C), D-α-tocopherol (vitamin E), and the like.

The culture temperature when inducing differentiation from endodermal cells to primitive gut tube cells is not particularly limited as long as the culture temperature is suitable for culturing the pluripotent stem cells that are used, but the culture temperature should generally be 30° C. to 40° C., preferably approximately 37° C.

The cells should preferably be cultured by using a CO₂ incubator or the like, in an atmosphere with a CO₂ concentration of approximately 1% to 10%, preferably 5%.

The induction of differentiation from endodermal cells into primitive gut tube (PGT) cells may be implemented in either an adhesion culture or a suspension culture, but is preferably in a suspension culture. The suspension culture may be performed under the suspension culture conditions described below. Furthermore, the mode thereof is not limited, and the cells may be suspension cultured after being adhered to a microcarrier or the like in advance, cell clumps composed only of cells may be suspension cultured, or a polymer such as collagen may be mixed into cell clumps.

The suspension culture may be a static culture using the viscosity of the culture medium or the like, a microwell having recesses and protrusions or the like, or may involve culturing the cells under conditions in which a liquid culture medium flows with the use of a spinner or the like. Preferably, the suspension culture involves culturing the cells under conditions in which a liquid culture medium flows. Culturing the cells under conditions in which a liquid culture medium flows preferably involves culturing the cells under conditions in which the liquid culture medium flows so as to promote cell aggregation. Examples of culturing cells under conditions in which the liquid culture medium flows so as to promote cell aggregation include culturing the cells under conditions in which the liquid culture medium flows so that stresses (centrifugal force and centripetal force) due to flow such as rotational flow and rocking flow cause the cells to gather at one point, and culturing the cells under conditions such that the liquid culture medium flows with linear reciprocating motion. The cells are particularly preferably cultured by using rotational flow and/or rocking flow.

The culture vessel used for the suspension culture is preferably a vessel with low cell adhesion to the inner surfaces of the vessel. Such vessels with low cell adhesion to the inner surfaces of the vessel include, for example, plates that have been surface-treated for hydrophilization with a biocompatible material. For example, Nunclon™ Sphera (Thermo Fisher Scientific) may be used, but there is no limitation thereon. Additionally, the shape of the culture vessel is not particularly limited, and examples include culture vessels in the shape of a dish, a flask, a well, a bag, a spinner flask, or the like.

The period of time over which the aggregates are formed is not particularly limited as long as the period exceeds 6 hours. Specifically, the aggregates are preferably formed over a period of 1 day, 2 days, 3 days, 4 days, 5 days, 6 days, 7 days, or 2 weeks, 3 weeks, 4 weeks, 5 weeks, 6 weeks, 7 weeks, or 8 weeks.

The suspension culture medium is not particularly limited as long as it contains components that allow pluripotent stem cells to proliferate. An mTeSR1 (Veritas Corporation) culture medium containing 1 to 100 μM of Y-27632 (Cayman) or Essential 8™ containing 1 to 100 μM of Y-27632 (Cayman) and 1 to 100 mg/mL of BSA, or the like may be used.

The conditions for stirring or rotating the suspension culture are not particularly limited as long as they are within a range allowing pluripotent stem cells to form aggregates in the suspension. The upper limit is preferably 200 rpm, more preferably 150 rpm, even more preferably 120 rpm, more preferably 100 rpm, more preferably 90 rpm, more preferably 80 rpm, even more preferably 70 rpm, more preferably 60 rpm, particularly preferably 50 rpm, and most preferably 45 rpm. The lower limit is preferably 1 rpm, more preferably 10 rpm, even more preferably 20 rpm, more preferably 30 rpm, more preferably 40 rpm, and particularly preferably 45 rpm. The rotation width in the case of a rotation culture is not particularly limited. The lower limit may, for example, be 1 mm, preferably 10 mm, more preferably 20 mm, and the most preferably 25 mm. The upper limit of the rotation width may, for example, be 200 mm, preferably 100 mm, preferably 50 mm, more preferably 30 mm, and most preferably 25 mm. The rotation radius in the case of a rotation culture is also not particularly limited, and is preferably set so that the rotation width is within the aforementioned range. The lower limit of the rotation radius may, for example, be 5 mm, preferably 10 mm, and the upper limit may, for example, be 100 mm, preferably 50 mm. The rotation culture conditions are preferably set to be in these ranges because these conditions make it easier for cell aggregates with appropriate dimensions to be produced.

Additionally, the suspension culture may be based on a rocking culture, in which a liquid culture medium is made to flow by rocking agitation. A rocking culture is implemented by rocking a culture vessel containing the liquid culture medium and cells in a plane substantially perpendicular to the horizontal plane. The rocking rate is not particularly limited, and the rocking can be performed with a frequency, for example, of 2 to 50 times, preferably of 4 to 25 times (one complete cycle being regarded as one time) per minute. The rocking angle is not particularly limited, and may, for example, be 0.1° to 20°, and more preferably 2° to 10°. The conditions of the rocking culture are preferably within these ranges because such conditions allow cell clumps with appropriate dimensions to be produced.

Furthermore, the cells may be cultured by agitation by means of a motion combining rotation and rocking as described above.

A suspension culture using a spinner flask-shaped culture vessel is a culture that involves agitating a liquid culture medium by using a stirring blade in the culture vessel. The rotation speed and the amount of the culture medium are not particularly limited. If a commercially available spinner flask-shaped culture vessel is used, then the manufacturer-recommended amount of the culture solution may be suitably used. For example, a spinner flask from ABLE Corporation or the like may be suitably used.

In the present invention, the seeding density of the cells in the suspension culture is not particularly limited as long as the seeding density allows the cells to form aggregates, but the seeding density is preferably 1×10⁵ to 1×10⁷ cells/mL. The seeding density of the cells is preferably 2×10⁵ cells/mL or more, 3×10⁵ cells/mL or more, 4×10⁵ cells/mL or more, or 5×10⁵ cells/mL or more, and is preferably 9×10⁶ cells/mL or less, 8×10⁶ cells/mL or less, 7×10⁶ cells/mL or less, 6×10⁶ cells/mL or less, 5×10⁶ cells/mL or less, 4×10⁶ cells/mL or less, 3×10⁶ cells/mL or less, 2×10⁶ cells/mL or less, 1.9×10⁶ cells/mL or less, 1.8×10⁶ cells/mL or less, 1.7×10⁶ cells/mL or less, 1.6×10⁶ cells/mL or less, or 1.5×10⁶ cells/mL or less. In particular, a cell density in the range from 5×10⁵ cells/mL to 1.5×10⁶ cells/mL is preferable.

The cell aggregates include hundreds to thousands of cells per aggregate. In the present invention, the size (diameter) of the cell aggregates is not particularly limited and may, for example, be 50 μm or larger, 55 μm or larger, 60 μm or larger, 65 μm or larger, 70 μm or larger, 80 μm or larger, 90 μm or larger, 100 μm or larger, 110 μm or larger, 120 μm or larger, 130 μm or larger, 140 μm or larger, or 150 μm or larger, and may be 1000 μm or smaller, 900 μm or smaller, 800 μm or smaller, 700 μm or smaller, 600 μm or smaller, 500 μm or smaller, or 400 μm or smaller. Cell aggregates having a diameter of 150 μm to 400 μm are favorable for the present invention. Cell aggregates having diameters outside the above range may be mixed together.

The “size (diameter) of the cell aggregates” in this case can be considered to refer to the dimensions of the widest portions of the cell clumps in an observed image when the cell aggregates are observed under a microscope.

The amount of the culture solution in the suspension culture can be appropriately adjusted in accordance with the culture vessel. For example, when a 12-well plate (the bottom surface area of each well in plan view being 3.5 cm²) is used, the amount may be 0.5 ml/well or more, and 1.5 ml/well or less, more preferably 1 ml/well. For example, when a 6-well plate (the bottom surface area of each well in plan view being 9.6 cm²) is used, the amount may be 1.5 mL/well or more, preferably 2 mL/well or more, and more preferably 3 mL/well or more, and may be 6.0 mL/well or less, preferably 5 mL/well or less, and more preferably 4 mL/well or less. For example, when a 125 mL Erlenmeyer flask (an Erlenmeyer flask having a capacity of 125 mL) is used, the amount may be 10 mL/vessel or more, preferably 15 mL/vessel or more, more preferably 20 mL/vessel or more, more preferably 25 mL/vessel or more, and more preferably 30 mL/vessel or more, and may be 50 mL/vessel or less, more preferably 45 mL/vessel or less, and more preferably 40 mL/vessel or less. For example, when a 500 mL Erlenmeyer flask (an Erlenmeyer flask having a capacity of 500 mL) is used, the amount may be 100 mL/vessel or more, preferably 105 mL/vessel or more, more preferably 110 mL/vessel or more, more preferably 115 mL/vessel or more, and more preferably 120 mL/vessel or more, and may be 150 mL/vessel or less, more preferably 145 mL/vessel or less, more preferably 140 mL/vessel or less, more preferably 135 mL/vessel or less, more preferably 130 mL/vessel or less, and more preferably 125 mL/vessel or less. For example, when a 1000 mL Erlenmeyer flask (an Erlenmeyer flask having a capacity of 1000 mL) is used, the amount may be 250 mL/vessel or more, preferably 260 mL/vessel or more, more preferably 270 mL/vessel or more, more preferably 280 mL/vessel or more, and more preferably 290 mL/vessel or more, and may be 350 mL/vessel or less, more preferably 340 mL/vessel or less, more preferably 330 mL/vessel or less, more preferably 320 mL/vessel or less, and more preferably 310 mL/vessel or less. For example, when a 2000 mL Erlenmeyer flask (an Erlenmeyer flask having a capacity of 2000 mL) is used, the amount may be 500 mL/vessel or more, more preferably 550 mL/vessel or more, and more preferably 600 mL/vessel or more, and may be 1000 mL/vessel or less, more preferably 900 mL/vessel or less, more preferably 800 mL/vessel or less, and more preferably 700 mL/vessel or less. For example, when a 3000 mL Erlenmeyer flask (an Erlenmeyer flask having a capacity of 3000 mL) is used, the amount may be 1000 mL/vessel or more, preferably 1100 mL/vessel or more, more preferably 1200 mL/vessel or more, more preferably 1300 mL/vessel or more, more preferably 1400 mL/vessel or more, and more preferably 1500 mL/vessel or more, and may be 2000 mL/vessel or less, more preferably 1900 mL/vessel or less, more preferably 1800 mL/vessel or less, more preferably 1700 mL/vessel or less, and more preferably 1600 mL/vessel or less. For example, when a 2 L culture bag (a disposable culture bag having a capacity of 2 L) is used, the amount may be 100 mL/bag or more, more preferably 200 mL/bag or more, more preferably 300 mL/bag or more, more preferably 400 mL/bag or more, more preferably 500 mL/bag or more, more preferably 600 mL/bag or more, more preferably 700 mL/bag or more, more preferably 800 mL/bag or more, more preferably 900 mL/bag or more, and more preferably 1000 mL/bag or more, and may be 2000 mL/bag or less, more preferably 1900 mL/bag or less, more preferably 1800 mL/bag or less, more preferably 1700 mL/bag or less, more preferably 1600 mL/bag or less, more preferably 1500 mL/bag or less, more preferably 1400 mL/bag or less, more preferably 1300 mL/bag or less, more preferably 1200 mL/bag or less, and more preferably 1100 mL/bag or less. For example, when a 10 L culture bag (a disposable culture bag having a capacity of 10 L) is used, the amount may be 500 mL/bag or more, more preferably 1 L/bag or more, more preferably 2 L/bag or more, more preferably 3 L/bag or more, more preferably 4 L/bag or more, and more preferably 5 L/bag or more, and may be 10 L/bag or less, more preferably 9 L/bag or less, more preferably 8 L/bag or less, more preferably 7 L/bag or less, and more preferably 6 L/bag or less. For example, when a 20 L culture bag (a disposable culture bag having a capacity of 20 L) is used, the amount may be 1 L/bag or more, more preferably 2 L/bag or more, more preferably 3 L/bag or more, more preferably 4 L/bag or more, more preferably 5 L/bag or more, more preferably 6 L/bag or more, more preferably 7 L/bag or more, more preferably 8 L/bag or more, more preferably 9 L/bag or more, and more preferably 10 L/bag or more, and may be 20 L/bag or less, more preferably 19 L/bag or less, more preferably 18 L/bag or less, more preferably 17 L/bag or less, more preferably 16 L/bag or less, more preferably 15 L/bag or less, more preferably 14 L/bag or less, more preferably 13 L/bag or less, more preferably 12 L/bag or less, and more preferably 11 L/bag or less. For example, when a 50 L culture bag (a disposable culture bag having a capacity of 50 L) is used, the amount may be 1 L/bag or more, more preferably 2 L/bag or more, more preferably 5 L/bag or more, more preferably 10 L/bag or more, more preferably 15 L/bag or more, more preferably 20 L/bag or more, and more preferably 25 L/bag or more, and may be 50 L/bag or less, more preferably 45 L/bag or less, more preferably 40 L/bag or less, more preferably 35 L/bag or less, and more preferably 30 L/bag or less. When the amount of the culture solution is within these ranges, cell aggregates of the appropriate size can be easily formed.

The capacity of the culture vessel that is used may be selected as appropriate and is not particularly limited, but in terms of the area, when seen in plan view, of the bottom surface of the portion in which the liquid culture medium is contained, the lower limit may, for example, be 0.32 cm², preferably 0.65 cm², more preferably 0.95 cm², even more preferably 1.9 cm², still more preferably 3.0 cm², 3.5 cm², 9.0 cm², or 9.6 cm², and the upper limit may, for example, be 1000 cm², preferably 500 cm², more preferably 300 cm², more preferably 150 cm², more preferably 75 cm², still more preferably 55 cm², even more preferably 25 cm², even more preferably 21 cm², and yet more preferably 9.6 cm², or 3.5 cm².

The culture period for inducing differentiation from endodermal cells to primitive gut tube cells is generally from 24 hours to 120 hours, and is preferably about 48 hours to 96 hours. For example, it may be 72 hours.

### [3] Primitive Gut Tube (PGT) Cells

Primitive gut tube cells form the foregut, the midgut and the hindgut. The midgut is connected to the yolk sac and the extraembryonic allantois branches from the hindgut. Additionally, the pharynx in the respiratory system is also formed from the foregut.

There are organs, such as the stomach and the intestines, into which the gut tubes directly differentiate, and those like the liver, the gall bladder, the pancreas, (the spleen (lymphoid organs)), and the like that are formed by budding from gut tubes. The differentiation from endodermal cells to primitive gut tube cells can be confirmed by measuring the expression levels of genes that are specific to primitive gut tube cells. Examples of genes that are specific to primitive gut tube cells include HNF-1β, HNF-4α and the like.

Primitive gut tube cells generally express at least one of HNF-1β and HNF-4α.

The gene sequence of HNF-1β (hepatocyte nuclear factor 1 beta) is registered in the gene database at the National Center for Biotechnology Information (see ID:6928).

The gene sequence of HNF-4a (octamer-binding transcription factor 4) is registered in the gene database at the National Center for Biotechnology Information (see ID:3172).

In the primitive gut tube cells of the present invention, it is preferable for gene expression related to the pathway “Biosynthesis of amino acids” (http://www.genome.jp/kegg-bin/show_pathway?map=hsa04015&show_description=show) to be elevated. For example, it is preferable for the expression level to be elevated, in comparison with primitive gut tube cells prepared by existing methods, for genes such as the N-acetylglutamate synthase (NAGS) gene (ENSEMBL_GENE_ID: ENSG00000161653); the aldolase, fructose-bisphosphate A (ALDOA) gene (ENSEMBL_GENE_ID: ENSG00000149925); the aldolase, fructose-bisphosphate C (ALDOC) gene (ENSEMBL_GENE_ID: ENSG00000109107); the aminoadipate aminotransferase (AADAT) gene (ENSEMBL_GENE_ID: ENSG00000109576); the argininosuccinate synthase 1 (ASS1) gene (ENSEMBL_GENE_ID: ENSG00000130707); the branched chain amino acid transaminase 1 (BCAT1) gene (ENSEMBL_GENE_ID: ENSG00000060982); the enolase 1 (ENO1) gene (ENSEMBL_GENE_ID: ENSG00000074800); the enolase 2 (ENO2) gene (ENSEMBL_GENE_ID: ENSG00000111674); the glutamate-ammonia ligase (GLUL) gene (ENSEMBL_GENE_ID: ENSG00000135821); the phosphofructokinase, liver type (PFKL) gene (ENSEMBL_GENE_ID: ENSG00000141959); the phosphofructokinase, platelet (PFKP) gene (ENSEMBL_GENE_ID: ENSG00000067057); the phosphoglycerate kinase 1 (PGK1) gene (ENSEMBL_GENE_ID: ENSG00000102144), the phosphoserine phosphatase (PSPH) gene (ENSEMBL_GENE_ID: ENSG00000146733), the pyrroline-5-carboxylate reductase 1 (PYCR1) gene (ENSEMBL_GENE_ID: ENSG00000183010), the pyruvate kinase, muscle (PKM) gene (ENSEMBL_GENE_ID: ENSG00000067225), the serine hydroxymethyltransferase 2 (SHMT2) gene (ENSEMBL_GENE_ID: ENSG00000182199), and the transketolase (TKT) gene (ENSEMBL_GENE_ID: ENSG00000163931). High expression levels of these genes mean that the enzymes involved in amino acid synthesis in the cells will increase and the biosynthesis of amino acids that serve as materials for proteins necessary for differentiation will become more active. Thus, the cells are in more appropriate state for inducing differentiation.

In the primitive gut tube cells of the present invention, it is preferable for gene expression related to the pathway “Rap1 signaling pathway” (http://www.genome.jp/kegg-bin/show_pathway?map=hsa04015&show_description=show) to be elevated. For example, it is preferable for the expression level to be elevated, in comparison with primitive gut tube cells prepared by existing methods, for genes such as the KIT proto-oncogene receptor tyrosine kinase (KIT) gene (ENSEMBL_GENE_ID: ENSG00000157404); the RAP1A, member of RAS oncogene family (RAP1A) gene (ENSEMBL_GENE_ID: ENSG00000116473); the Rap guanine nucleotide exchange factor 4 (RAPGEF4) gene (ENSEMBL_GENE_ID: ENSG00000091428); the adenylate cyclase 7 (ADCY7) gene (ENSEMBL_GENE_ID: ENSG00000121281); the adenylate cyclase 8 (ADCY8) gene (ENSEMBL_GENE_ID: ENSG00000155897); the afadin, adherens junction formation factor (AFDN) gene (ENSEMBL_GENE_ID: ENSG00000130396); the amyloid beta precursor protein-binding family B member 1-interacting protein (APBB1IP) gene (ENSEMBL_GENE_ID: ENSG00000077420); the angiopoietin 1 (ANGPT1) gene (ENSEMBL_GENE_ID: ENSG00000154188); the calmodulin 1 (CALM1) gene (ENSEMBL_GENE_ID: ENSG00000198668); the ephrin A1 (EFNA1) gene (ENSEMBL_GENE_ID: ENSG00000169242); the ephrin A3 (EFNA3) gene (ENSEMBL_GENE_ID: ENSG00000143590); the ephrin A5 (EFNA5) gene (ENSEMBL_GENE_ID: ENSG0000018434); the fibroblast growth factor 11 (FGF11) gene (ENSEMBL_GENE_ID: ENSG00000161958); the fibroblast growth factor receptor 3 (FGFR3) gene (ENSEMBL_GENE_ID: ENSG00000068078); the fibroblast growth factor receptor 4 (FGFR4) gene (ENSEMBL_GENE_ID: ENSG00000160867); the glutamate ionotropic receptor NMDA type subunit 2A (GRIN2A) gene (ENSEMBL_GENE_ID: ENSG00000183454); the insulin-like growth factor 1 (IGF1) gene (ENSEMBL_GENE_ID: ENSG00000017427); the membrane-associated guanylate kinase, WW and PDZ domain containing 3 (MAGI3) gene (ENSEMBL_GENE_ID: ENSG00000081026); the phosphoinositide-3-kinase regulatory subunit 1 (PIK3R1) gene (ENSEMBL_GENE_ID: ENSG00000145675); the phosphoinositide-3-kinase regulatory subunit 5 (PIK3R5) gene (ENSEMBL_GENE_ID: ENSG00000141506); the phospholipase C beta 1 (PLCB1) gene (ENSEMBL_GENE_ID: ENSG00000182621); the phospholipase C epsilon 1 (PLCE1) gene (ENSEMBL_GENE_ID: ENSG00000138193); the placental growth factor (PGF) gene (ENSEMBL_GENE_ID: ENSG0000011963); the platelet-derived growth factor D (PDGFD) gene (ENSEMBL_GENE_ID: ENSG00000170962); the platelet-derived growth factor receptor alpha (PDGFRA) gene (ENSEMBL_GENE_ID: ENSG00000134853); the regulator of G-protein signaling 14 (RGS14) gene (ENSEMBL_GENE_ID: ENSG00000169220); the signal-induced proliferation-associated 1-like 2 (SIPA1L2) gene (ENSEMBL_GENE_ID: ENSG00000116991); the talin 2 (TLN2) gene (ENSEMBL_GENE_ID: ENSG00000171914); and the vascular endothelial growth factor C (VEGFC) gene (ENSEMBL_GENE_ID: ENSG00000150630). High expression levels of these genes can be considered to contribute to increases in cell adhesion, the three-dimensional structuralization of tissues, and the like. Thus, the cells can be considered to be in more appropriate state for inducing differentiation.

In the primitive gut tube cells of the present invention, it is preferable for gene expression related to the pathway “Pathways in cancer” (http://www.genome.jp/kegg-bin/show_pathway?map=hsa05200&show_description=show) to be elevated. For example, it is preferable for the expression level to be elevated, in comparison with primitive gut tube cells prepared by existing methods, for genes such as the A-Raf proto-oncogene, serine/threonine kinase (ARAF) gene (ENSEMBL_GENE_ID: ENSG00000078061); the BCR, RhoGEF and GTPase activating protein (BCR) gene (ENSEMBL_GENE_ID: ENSG0000018671); the C-X-C motif chemokine receptor 4 (CXCR4) gene (ENSEMBL_GENE_ID: ENSG0000012196); the CCAAT/enhancer-binding protein alpha (CEBPA) gene (ENSEMBL_GENE_ID: ENSG00000245848); the Cbl proto-oncogene C (CBLC) gene (ENSEMBL_GENE_ID: ENSG00000142273); the KIT proto-oncogene receptor tyrosine kinase (KIT) gene (ENSEMBL_GENE_ID: ENSG00000157404); the MDS1 and EVI1 complex locus (MECOM) gene (ENSEMBL_GENE_ID: ENSG00000085276); the SMAD family member 3 (SMAD3) gene (ENSEMBL_GENE_ID: ENSG00000166949); the adenylate cyclase 7 (ADCY7) gene (ENSEMBL_GENE_ID: ENSG00000121281); the adenylate cyclase 8 (ADCY8) gene (ENSEMBL_GENE_ID: ENSG00000155897); the catenin alpha 3 (CTNNA3) gene (ENSEMBL_GENE_ID: ENSG00000183230); the collagen type IV alpha 3 chain (COL4A3) gene (ENSEMBL_GENE_ID: ENSG00000169031); the collagen type IV alpha 5 chain (COL4A5) gene (ENSEMBL_GENE_ID: ENSG00000188153); the collagen type IV alpha 6 chain (COL4A6) gene (ENSEMBL_GENE_ID: ENSG00000197565); the cyclin D1(CCND1) gene (ENSEMBL_GENE_ID: ENSG00000110092); the egl-9 family hypoxia inducible factor 1 (EGLN1) gene (ENSEMBL_GENE_ID: ENSG0000013576); the endothelial PAS domain protein 1 (EPAS1) gene (ENSEMBL_GENE_ID: ENSG00000116016); the endothelin receptor type A (EDNRA) gene (ENSEMBL_GENE_ID: ENSG00000151617); the fibronectin 1 (FN1) gene (ENSEMBL_GENE_ID: ENSG00000115414); the frizzled class receptor 1 (FZD1) gene (ENSEMBL_GENE_ID: ENSG00000157240); the frizzled class receptor 2 (FZD2) gene (ENSEMBL_GENE_ID: ENSG00000180340); the laminin subunit beta 1 (LAMB1) gene (ENSEMBL_GENE_ID: ENSG00000091136); the lysophosphatidic acid receptor 6 (LPAR6) gene (ENSEMBL_GENE_ID: ENSG00000139679); the mitogen-activated protein kinase 10 (MAPK10) gene (ENSEMBL_GENE_ID: ENSG00000109339); the patched 1 (PTCH1) gene (ENSEMBL_GENE_ID: ENSG00000185920); the peroxisome proliferator-activated receptor gamma (PPARG) gene (ENSEMBL_GENE_ID: ENSG00000132170); the phosphoinositide-3-kinase regulatory subunit 1 (PIK3R1) gene (ENSEMBL_GENE_ID: ENSG00000145675); the phosphoinositide-3-kinase regulatory subunit 5 (PIK3R5) gene (ENSEMBL_GENE_ID: ENSG00000141506); the phospholipase C beta 1 (PLCB1) gene (ENSEMBL_GENE_ID: ENSG00000182621); the phospholipase C gamma 2 (PLCG2) gene (ENSEMBL_GENE_ID: ENSG00000197943); the prostaglandin E receptor 2 (PTGER2) gene (ENSEMBL_GENE_ID: ENSG00000125384); the protein inhibitor of activated STAT 2 (PIAS2) gene (ENSEMBL_GENE_ID: ENSG00000078043); the retinoid X receptor alpha (RXRA) gene (ENSEMBL_GENE_ID: ENSG00000186350); the solute carrier family 2 member 1 (SLC2A1) gene (ENSEMBL_GENE_ID: ENSG00000117394); the transforming growth factor beta 1 (TGFB1) gene (ENSEMBL_GENE_ID: ENSG00000105329); the transforming growth factor beta receptor 2 (TGFBR2) gene (ENSEMBL_GENE_ID: ENSG00000163513); the tropomyosin 3 (TPM3) gene (ENSEMBL_GENE_ID: ENSG00000143549); and the vascular endothelial growth factor C (VEGFC) gene (ENSEMBL_GENE_ID: ENSG00000150630). High expression levels of these genes can be considered to indicate the possibility that various signaling pathways necessary for differentiation will be activated. Thus, the cells can be considered to be in more appropriate state for inducing differentiation.

In the primitive gut tube cells of the present invention, it is preferable for gene expression related to the pathway “p53 signaling pathway” (http://www.genome.jp/kegg-bin/show_pathway?map=hsa04115&show_description=show) to be reduced. For example, it is preferable for the expression level to be reduced, in comparison with primitive gut tube cells prepared by existing methods, for genes such as the BCL2-associated X, apoptosis regulator (BAX) gene (ENSEMBL_GENE_ID: ENSG00000087088); the Fas cell surface death receptor (FAS) gene (ENSEMBL_GENE_ID: ENSG00000026103); the MDM2 proto-oncogene (MDM2) gene (ENSEMBL_GENE_ID: ENSG00000135679); the PERP, TP53 apoptosis effector (PERP) gene (ENSEMBL_GENE_ID: ENSG00000112378); the STEAP3 metalloreductase (STEAP3) gene (ENSEMBL_GENE_ID: ENSG00000115107); the caspase 3 (CASP3) gene (ENSEMBL_GENE_ID: ENSG00000164305); the caspase 8 (CASP8) gene (ENSEMBL_GENE_ID: ENSG00000064012); the cyclin D2 (CCND2) gene (ENSEMBL_GENE_ID: ENSG00000118971); the cyclin E2 (CCNE2) gene (ENSEMBL_GENE_ID: ENSG00000175305); the cyclin dependent kinase 1 (CDK1) gene (ENSEMBL_GENE_ID: ENSG00000170312); the cyclin dependent kinase 6 (CDK6) gene (ENSEMBL_GENE_ID: ENSG00000105810); the cyclin-dependent kinase inhibitor 1A (CDKN1A) gene (ENSEMBL_GENE_ID: ENSG00000124762); the damage-specific DNA-binding protein 2 (DDB2) gene (ENSEMBL_GENE_ID: ENSG00000134574); the phorbol-12-myristate-13-acetate-induced protein 1 (PMAIP1) gene (ENSEMBL_GENE_ID: ENSG00000141682); the protein phosphatase, Mg²⁺/Mn²⁺-dependent 1D (PPM1D) gene (ENSEMBL_GENE_ID: ENSG00000170836); the reprimo, TP53-dependent G2 arrest mediator candidate (RPRM) gene (ENSEMBL_GENE_ID: ENSG00000177519); the ribonucleotide reductase regulatory TP53-inducible subunit M2B (RRM2B) gene (ENSEMBL_GENE_ID: ENSG00000048392); the serpin family B member 5 (SERPINB5) gene (ENSEMBL_GENE_ID: ENSG00000206075); the serpin family E member 1 (SERPINE1) gene (ENSEMBL_GENE_ID: ENSG00000106366); the sestrin 2 (SESN2) gene (ENSEMBL_GENE_ID: ENSG00000130766); the stratifin (SFN) gene (ENSEMBL_GENE_ID: ENSG00000175793); and the zinc finger matrin-type 3 (ZMAT3) gene (ENSEMBL_GENE_ID: ENSG00000172667). Low expression levels of these genes can be considered to indicate the possibility that cell death is suppressed. Thus, the cells can be considered to be in more appropriate state for inducing differentiation.

Examples of the primitive gut tube (PGT) cells of the present invention include cells in which the expression of at least one gene selected from the group consisting of the KIT gene, the RAP1A gene, the FGF11 gene, and the FGFR4 gene is elevated, and/or the expression of at least one gene selected from the group consisting of the MDM2 gene, the CASP3 gene, and the CDK1 gene is reduced in comparison with primitive gut tube (PGT) cells produced by the method (i.e., culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells) in Comparative Example 5 explained below.

In the primitive gut tube (PGT) cells of the present invention, the expression of at least one gene selected from the group consisting of the IGFBP3 gene, the PTGDR gene, the LOX gene, the PAPPA gene, and the RAB31 gene is preferably elevated in comparison with primitive gut tube (PGT) cells produced by the method in Comparative Example 5.

In the primitive gut tube (PGT) cells of the present invention, the expression of at least one gene selected from the group consisting of the ANGPT2 gene, the CD47 gene, the CDC42EP3 gene, the CLDN18 gene, the CLIC5 gene, the PHLDA1 gene, and the SKAP2 gene is preferably reduced in comparison with primitive gut tube (PGT) cells produced by the method in Comparative Example 5.

Other examples of the primitive gut tube (PGT) cells of the present invention include primitive gut tube (PGT) cells in which the expression of at least one gene selected from the group consisting of the IGFBP3 gene, the PTGDR gene, and the PAPPA gene is elevated and/or the expression of at least one gene selected from the group consisting of the ANGPT2 gene and the FRZB gene is reduced in comparison with endodermal cells that have been induced to differentiate from pluripotent stem cells.

Yet other examples of the primitive gut tube (PGT) cells of the present invention include primitive gut tube (PGT) cells in which the expression of at least one or more genes selected from the group consisting of the IGFBP3 gene, the PTGDR gene, the LOX gene, the PAPPA gene, and the RAB31 gene is elevated and/or the expression of at least one or more genes selected from the group consisting of the ANGPT2 gene, the BMPR1B gene, the CD47 gene, the CDC42EP3 gene, the CLDN18 gene, the CLIC5 gene, the FRZB gene, the IGF2 gene, the PHLDA1 gene, and the SKAP2 gene is reduced in comparison with primitive gut tube (PGT) cells produced by culturing, in the presence of a bone morphogenetic protein (BMP) signaling inhibitor, a TGF-β signaling inhibitor, and a hedgehog (HH) signaling inhibitor, endodermal cells that have been induced to differentiate from pluripotent stem cells.

Furthermore, the present invention provides a cell population including primitive gut tube cells, wherein the cell population has the cell properties in (a) to (d) indicated below:

(a) in the cell population, the relative expression level of the FGF11 gene with respect to the expression level of the β-Actin gene is 0.01 or higher;
(b) in the cell population, the relative expression level of the FGFR4 gene with respect to the expression level of the β-Actin gene is 0.03 or higher;
(c) in the cell population, the relative expression level of the CASP3 gene with respect to the expression level of the β-Actin gene is 0.006 or lower; and
(d) in the cell population, the relative expression level of the CDK1 gene with respect to the expression level of the β-Actin gene is 0.02 or lower.

In the aforementioned cell population, it is sufficient for one or more of the following conditions to be satisfied:

the relative expression level of the RAP1A gene with respect to the expression level of the β-Actin gene is 0.03 or higher;
the relative expression level of the KIT gene with respect to the expression level of the (3-Actin gene is 0.05 or higher; or
the relative expression level of the MDM2 gene with respect to the expression level of the β-Actin gene is 0.03 or lower.

In the aforementioned cell population, the relative expression level of the IGFBP3 gene with respect to the expression level of the OAZ1 gene may be 10 or higher, the relative expression level of the PTGDR gene with respect to the expression level of the OAZ1 gene may be 0.6 or higher, the relative expression level of the LOX gene with respect to the expression level of the OAZ1 gene may be 0.6 or higher, the relative expression level of the PAPPA gene with respect to the expression level of the OAZ1 gene may be 0.01 or higher, and the relative expression level of the RAB31 gene with respect to the expression level of the OAZ1 gene may be 0.2 or higher.

In the aforementioned cell population, the relative expression level of the ANGPT2 gene with respect to the expression level of the OAZ1 gene may be 0.0002 or lower, the relative expression level of the CD47 gene with respect to the expression level of the OAZ1 gene may be 0.02 or lower, the relative expression level of the CDC42EP3 gene with respect to the expression level of the OAZ1 gene may be 0.03 or lower, the relative expression level of the CLDN18 gene with respect to the expression level of the OAZ1 gene may be 0.006 or lower, the relative expression level of the CLIC5 gene with respect to the expression level of the OAZ1 gene may be 0.0001 or lower, the relative expression level of the PHLDA1 gene with respect to the expression level of the OAZ1 gene may be 0.2 or lower, and the relative expression level of the SKAP2 gene with respect to the expression level of the OAZ1 gene may be 0.01 or lower.

The relative expression level of the FGF11 (ENSEMBL_GENE_ID: ENSG00000161958) gene with respect to the expression level of the β-Actin (NCBI Gene ID: 60) gene is 0.01 or higher, and may favorably be 0.02 or higher, 0.03 or higher, 0.04 or higher, 0.05 or higher, 0.06 or higher, 0.07 or higher, 0.08 or higher, 0.09 or higher, or 0.1 or higher. The FGF11 gene is a gene involved in the “Rap1 signaling pathway”. Therefore, the fact that the relative expression level of the FGF11 gene with respect to the β-Actin gene is 0.01 or higher can be considered to contribute to increased cell adhesion and three-dimensional structuralization of tissues. Thus, the cells can be considered to be in more appropriate state for inducing differentiation.

The relative expression level of the FGFR4 (ENSEMBL_GENE_ID: ENSG00000160867) gene with respect to the expression level of the β-Actin gene is 0.03 or higher, and may favorably be 0.04 or higher, 0.05 or higher, or 0.1 or higher. The FGFR4 gene is a gene involved in the “Rap1 signaling pathway”. Therefore, the fact that the relative expression level of the FGFR4 gene with respect to the β-Actin gene is 0.03 or higher can be considered to contribute to increased cell adhesion and three-dimensional structuralization of tissues. Thus, the cells can be considered to be in more appropriate state for inducing differentiation.

The relative expression level of the CASP3 (ENSEMBL_GENE_ID: ENSG00000164305) gene with respect to the expression level of the β-Actin gene is 0.006 or lower, and may favorably be 0.005 or lower, 0.004 or lower, 0.003 or lower, 0.002 or lower, 0.001 or lower, 0.0005 or lower, or 0.0001 or lower. The CASP3 gene is a gene involved in the “p53 signaling pathway”. Therefore, the fact that the relative expression level of the CASP3 gene with respect to the β-Actin gene is 0.006 or lower can be considered to indicate that there is a possibility that cell death will be suppressed. Thus, the cells can be considered to be in more appropriate state for inducing differentiation.

The relative expression level of the CDK1 (ENSEMBL_GENE_ID: ENSG00000170312) gene with respect to the expression level of the β-Actin gene is 0.02 or lower, and may favorably be 0.01 or lower, 0.005 or lower, or 0.001 or lower. The CDK1 gene is a gene involved in the “p53 signaling pathway”. Therefore, the fact that the relative expression level of the CDK1 gene with respect to the β-Actin gene is 0.02 or lower can be considered to indicate that there is a possibility that cell death will be suppressed. Thus, the cells can be considered to be in more appropriate state for inducing differentiation.

The relative expression level of the RAP1A (ENSEMBL_GENE_ID: ENSG00000116473) gene with respect to the expression level of the β-Actin gene is 0.03 or higher, and may favorably be 0.04 or higher, 0.05 or higher, 0.06 or higher, 0.07 or higher, 0.08 or higher, 0.09 or higher, or 0.1 or higher. The RAP1A gene is a gene involved in the “Rap1 signaling pathway”. Therefore, the fact that the relative expression level of the RAP1A gene with respect to the β-Actin gene is 0.03 or higher can be considered to contribute to increased cell adhesion and three-dimensional structuralization of tissues. Thus, the cells can be considered to be in more appropriate state for inducing differentiation.

The relative expression level of the KIT (ENSEMBL_GENE_ID: ENSG00000157404) gene with respect to the expression level of the β-Actin gene is 0.05 or higher, and may favorably be 0.06 or higher, 0.07 or higher, 0.08 or higher, 0.09 or higher, 0.1 or higher, or 0.5 or higher. The KIT gene is a gene involved in the “Rap1 signaling pathway”. Therefore, the fact that the relative expression level of the KIT gene with respect to the β-Actin gene is 0.05 or higher can be considered to contribute to increased cell adhesion and three-dimensional structuralization of tissues. Thus, the cells can be considered to be in more appropriate state for inducing differentiation.

The relative expression level of the MDM2 (MDM2 proto-oncogene; ENSEMBL_GENE_ID: ENSG00000135679) gene with respect to the expression level of the β-Actin gene is 0.03 or lower, and may favorably be 0.02 or lower, 0.01 or lower, 0.005 or lower, or 0.001 or lower. The MDM2 gene is a gene involved in the “p53 signaling pathway”. Therefore, the fact that the relative expression level of the MDM2 gene with respect to the β-Actin gene is 0.03 or lower can be considered to indicate that there is a possibility that cell death will be suppressed. Thus, the cells can be considered to be in more appropriate state for inducing differentiation.

The relative expression level of the IGFBP3 (Insulin-like growth factor-binding protein 3; NCBI Gene ID: 3486) gene with respect to the expression level of the OAZ1 (ornithine decarboxylase antizyme 1; for the gene sequence, see ID: 4946 registered in the gene database at the National Center for Biotechnology Information) gene may be 10 or higher, and may favorably be 11 or higher, 12 or higher, 13 or higher, 14 or higher, 15 or higher, 16 or higher, 17 or higher, 18 or higher, 19 or higher, 20 or higher, 30 or higher, 40 or higher, 50 or higher, 60 or higher, 70 or higher, 80 or higher, 90 or higher, or 100 or higher. The IGFBP3 gene is highly expressed in the primitive gut tube cells of the present invention, and thus can be considered to be usable as a positive marker gene for the primitive gut tube cells.

The relative expression level of the PTGDR (Prostaglandin D2 receptor; NCBI Gene ID: 5729) gene with respect to the expression level of the OAZ1 gene may be 0.6 or higher, and may favorably be 0.7 or higher, 0.8 or higher, 0.9 or higher, 1 or higher, 2 or higher, 3 or higher, 4 or higher, 5 or higher, 6 or higher, 7 or higher, 8 or higher, 9 or higher, 10 or higher, 20 or higher, 30 or higher, 40 or higher, 50 or higher, 60 or higher, 70 or higher, 80 or higher, 90 or higher, or 100 or higher. The PTGDR gene is highly expressed in the primitive gut tube cells of the present invention, and thus can be considered to be usable as a positive marker gene for the primitive gut tube cells.

The relative expression level of the LOX (Lysyl oxidase; NCBI Gene ID: 4015) gene with respect to the expression level of the OAZ1 gene may be 0.6 or higher, and may favorably be 0.7 or higher, 0.8 or higher, 0.9 or higher, 1 or higher, 2 or higher, 3 or higher, 4 or higher, 5 or higher, 6 or higher, 7 or higher, 8 or higher, 9 or higher, 10 or higher, 20 or higher, 30 or higher, 40 or higher, 50 or higher, 60 or higher, 70 or higher, 80 or higher, 90 or higher, or 100 or higher. The LOX gene is highly expressed in the primitive gut tube cells of the present invention, and thus can be considered to be usable as a positive marker gene for the primitive gut tube cells.

The relative expression level of the PAPPA (Pappalysin 1; NCBI Gene ID: 5069) gene with respect to the expression level of the OAZ1 gene may be 0.01 or higher, and may favorably be 0.02 or higher, 0.03 or higher, 0.04 or higher, 0.05 or higher, 0.06 or higher, 0.07 or higher, 0.08 or higher, 0.09 or higher, 0.1 or higher, 0.2 or higher, 0.3 or higher, 0.4 or higher, 0.5 or higher, 0.6 or higher, 0.7 or higher, 0.8 or higher, 0.9 or higher, 1 or higher, 2 or higher, 3 or higher, 4 or higher, 5 or higher, 6 or higher, 7 or higher, 8 or higher, 9 or higher, 10 or higher, 20 or higher, 30 or higher, 40 or higher, 50 or higher, 60 or higher, 70 or higher, 80 or higher, 90 or higher, or 100 or higher. The PAPPA gene is highly expressed in the primitive gut tube cells of the present invention, and thus can be considered to be usable as a positive marker gene for the primitive gut tube cells.

The relative expression level of the RAB31 (RAB31, member RAS oncogene family; NCBI Gene ID: 11031) gene with respect to the expression level of the OAZ1 gene may be 0.2 or higher, and may favorably be 0.3 or higher, 0.4 or higher, 0.5 or higher, 0.6 or higher, 0.7 or higher, 0.8 or higher, 0.9 or higher, 1 or higher, 2 or higher, 3 or higher, 4 or higher, 5 or higher, 6 or higher, 7 or higher, 8 or higher, 9 or higher, 10 or higher, 20 or higher, 30 or higher, 40 or higher, 50 or higher, 60 or higher, 70 or higher, 80 or higher, 90 or higher, or 100 or higher. The RAB31 gene is highly expressed in the primitive gut tube cells of the present invention, and thus can be considered to be usable as a positive marker gene for the primitive gut tube cells.

The relative expression level of the ANGPT2 (Angiopoietin 2; NCBI Gene ID: 285) gene with respect to the expression level of the OAZ1 gene may be 0.0002 or lower, and may favorably be 0.0001 or lower, 0.00009 or lower, 0.00008 or lower, 0.00007 or lower, 0.00006 or lower, 0.00005 or lower, 0.00004 or lower, 0.00003 or lower, 0.00002 or lower, or 0.00001 or lower. The ANGPT2 gene is lowly expressed in the primitive gut tube cells of the present invention, and thus can be considered to be usable as a negative marker gene for the primitive gut tube cells.

The relative expression level of the CD47 (CD47 molecule; NCBI Gene ID: 961) gene with respect to the expression level of the OAZ1 gene may be 0.02 or lower, and may favorably be 0.01 or lower, 0.009 or lower, 0.008 or lower, 0.007 or lower, 0.006 or lower, 0.005 or lower, 0.004 or lower, 0.003 or lower, 0.002 or lower, or 0.001 or lower. The CD47 gene is lowly expressed in the primitive gut tube cells of the present invention, and thus can be considered to be usable as a negative marker gene for the primitive gut tube cells.

The relative expression level of the CDC42EP3 (CDC42 effector protein 3; NCBI Gene ID: 10602) gene with respect to the expression level of the OAZ1 gene may be 0.03 or lower, and may favorably be 0.02 or lower, 0.01 or lower, 0.009 or lower, 0.008 or lower, 0.007 or lower, 0.006 or lower, 0.005 or lower, 0.004 or lower, 0.003 or lower, 0.002 or lower, or 0.001 or lower. The CDC42EP3 gene is lowly expressed in the primitive gut tube cells of the present invention, and thus can be considered to be usable as a negative marker gene for the primitive gut tube cells.

The relative expression level of the CLDN18 (Claudin 18; NCBI Gene ID: 51208) gene with respect to the expression level of the OAZ1 gene may be 0.006 or lower, and may favorably be 0.005 or lower, 0.004 or lower, 0.003 or lower, 0.002 or lower, 0.001 or lower, 0.0009 or lower, 0.0008 or lower, 0.0007 or lower, 0.0006 or lower, 0.0005 or lower, 0.0004 or lower, 0.0003 or lower, 0.0002 or lower, or 0.0001 or lower. The CLDN18 gene is lowly expressed in the primitive gut tube cells of the present invention, and thus can be considered to be usable as a negative marker gene for the primitive gut tube cells.

The relative expression level of the CLIC5 (Chloride intracellular channel 5; NCBI Gene ID: 53405) gene with respect to the expression level of the OAZ1 gene may be 0.0001 or lower, and may favorably be 0.00009 or lower, 0.00008 or lower, 0.00007 or lower, 0.00006 or lower, 0.00005 or lower, 0.00004 or lower, 0.00003 or lower, 0.00002 or lower, or 0.00001 or lower. The CLIC5 gene is lowly expressed in the primitive gut tube cells of the present invention, and thus can be considered to be usable as a negative marker gene for the primitive gut tube cells.

The relative expression level of the PHLDA1 (Pleckstrin homology-like domain family A member 1; NCBI Gene ID: 22822) gene with respect to the expression level of the OAZ1 gene may be 0.2 or lower, and may favorably be 0.1 or lower, 0.09 or lower, 0.08 or lower, 0.07 or lower, 0.06 or lower, 0.05 or lower, 0.04 or lower, 0.03 or lower, 0.02 or lower, 0.01 or lower, 0.009 or lower, 0.008 or lower, 0.007 or lower, 0.006 or lower, 0.005 or lower, 0.004 or lower, 0.003 or lower, 0.002 or lower, or 0.001 or lower. The PHLDA1 gene is lowly expressed in the primitive gut tube cells of the present invention, and thus can be considered to be usable as a negative marker gene for the primitive gut tube cells.

The relative expression level of the SKAP2 (Src kinase-associated phosphoprotein 2; NCBI Gene ID: 8935) gene with respect to the expression level of the OAZ1 gene may be 0.01 or lower, and may favorably be 0.009 or lower, 0.008 or lower, 0.007 or lower, 0.006 or lower, 0.005 or lower, 0.004 or lower, 0.003 or lower, 0.002 or lower, 0.001 or lower, 0.0009 or lower, 0.0008 or lower, 0.0007 or lower, 0.0006 or lower, 0.0005 or lower, 0.0004 or lower, 0.0003 or lower, 0.0002 or lower, or 0.0001 or lower. The SKAP2 gene is lowly expressed in the primitive gut tube cells of the present invention, and thus can be considered to be usable as a negative marker gene for the primitive gut tube cells.

The relative expression level of the FRZB (fizzled-related protein; NCBI Gene ID: 2487) gene with respect to the expression level of the OAZ1 gene may be 0.085 or lower, and may favorably be 0.08 or lower, 0.07 or lower, 0.06 or lower, 0.05 or lower, 0.04 or lower, 0.03 or lower, 0.02 or lower, 0.01 or lower, 0.009 or lower, 0.008 or lower, 0.007 or lower, 0.006 or lower, 0.005 or lower, 0.004 or lower, 0.003 or lower, 0.002 or lower, or 0.001 or lower. The FRZB gene is lowly expressed in the primitive gut tube cells of the present invention, and thus can be considered to be usable as a negative marker gene for the primitive gut tube cells.

### [4] Induced Differentiation to Pancreatic β Cells

### <Pancreatic β Cells>

Pancreatic β cells are cells differentiated from pancreatic endocrine precursor cells, and are cells that secrete insulin. The differentiation from pancreatic endocrine precursor cells to pancreatic β cells can be confirmed by measuring the expression levels of genes specific to pancreatic β cells. Examples of genes specific to pancreatic β cells include insulin, NKX6.1, MAFA, PDX1, and the like.

### <Induced Differentiation to Pancreatic β Cells>

The induced differentiation from endodermal cells to pancreatic β cells is generally performed in the sequence from endodermal cells (definitive endoderm: DE) to primitive intestinal cells (Primitive Gut Tube: PGT) to posterior foregut cells (Posterior Foregut: PFG) to pancreatic progenitor cells (PP) to endocrine precursor cells (EP) to pancreatic β cells (pancreatic β cells: β).

The culture temperature for inducing differentiation from endodermal cells into pancreatic β cells is not particularly limited as long as it is a culture temperature suitable for culturing the pluripotent stem cells to be used. Generally, the temperature is 30° C. to 40° C., and is preferably about 37° C.

The cells are preferably cultured by using a CO₂ incubator or the like in an atmosphere with a CO₂ concentration of about 1% to 10%, preferably 5%.

The induced differentiation from endodermal cells to primitive gut tube cells is performed in the manner described above in the present description.

The culture medium used for the induced differentiation from primitive gut tube cells to posterior foregut cells may be a culture medium prepared by adding antibiotics (penicillin and streptomycin), NEAA (non-essential amino acids), B27 supplement, EC23, and SANT1 to a basal medium (for example, a DMEM medium or the like).

The culture period for the induced differentiation from primitive gut tube cells to posterior foregut cells is generally 48 hours to 144 hours, preferably about 72 hours to 120 hours.

The culture medium used for the induced differentiation from posterior foregut cells to pancreatic progenitor cells may be a culture medium prepared by adding antibiotics (penicillin and streptomycin), NEAA (non-essential amino acids), FGF-10, B27 supplement, EC23, ALK5 inhibitor II, and Indolactam V to a basal medium (for example, a DMEM medium or the like).

The culture period for the induced differentiation from posterior foregut cells to pancreatic progenitor cells is generally 24 hours to 120 hours, preferably about 48 hours to 96 hours.

The culture medium used for the induced differentiation from pancreatic progenitor cells to endocrine precursor cells may be a culture medium prepared by adding antibiotics (penicillin and streptomycin), B27 supplement, EC23, SANT1, ALK5 inhibitor II, and Excedin-4 to a basal medium (for example, an advanced DMEM medium or the like).

The culture period for the induced differentiation from pancreatic progenitor cells to endocrine precursor cells is generally 24 hours to 120 hours, preferably about 48 hours to 96 hours.

The culture medium used for the induced differentiation from endocrine precursor cells to pancreatic β cells may be a culture medium prepared by adding antibiotics (penicillin and streptomycin), B27 supplement, BMP-4, HGF, IGF-1, ALK5 inhibitor II, Excedin-4, nicotinamide, and forskolin to a basal medium (for example, an advanced DMEM medium or the like).

The differentiation to pancreatic β cells can be confirmed by measuring the expression levels of genes specific to pancreatic β cells. Examples of genes that are specific to pancreatic β cells include INS (insulin), NKX6.1 (NK6 homeobox 1), and the like.

The gene sequence of INS is registered (ID: 3630) in the gene database at the National Center for Biotechnology Information, and the gene sequence of NKX6.1 is registered (ID: 4825) in the gene database at the National Center for Biotechnology Information.

The culture period for the induced differentiation from endocrine precursor cells to pancreatic β cells is generally about 96 hours to 240 hours.

The pancreatic β cells obtained by the abovementioned method have high insulin secretory capacity and can provide high therapeutic effects for diabetes. In other words, when the method of the present invention is used to obtain pancreatic β cells (sometimes referred to as insulin-producing cells), they can be used to treat diabetes by transplanting the cells with a catheter or the like, or transplanting the cells sealed in an immunoisolation device or the like. Additionally, by obtaining pancreatic cells that are metabolic, such as pancreatic β cells, they can be used to treat type I diabetes by directly injecting insulin produced by the pancreatic β cells.

Hereinafter, the method for producing endodermal cells from pluripotent stem cells will be explained. The method for inducing differentiation from pluripotent stem cells to endodermal cells may be any method that is conventionally known, and is not particularly limited to the specific embodiments indicated below.

### [5] Maintenance Culture of Pluripotent Stem Cells

In the production method of the present invention, endodermal cells that have been induced to differentiate by culturing pluripotent stem cells are used.

The undifferentiated state of the pluripotent stem cells before the induced differentiation to endodermal cells is preferably maintained by using an undifferentiated-state maintenance medium. A culture in which the undifferentiated state of pluripotent stem cells is maintained by using an undifferentiated-state maintenance medium is also called a maintenance culture of pluripotent stem cells.

The undifferentiated-state maintenance medium is not particularly limited as long as it is a culture medium that allows the undifferentiated state of pluripotent stem cells to be maintained. Examples include a culture medium containing a leukemia inhibitory factor that is known to have the property of maintaining the undifferentiated state of mouse embryonic stem cells and mouse induced pluripotent stem cells, a culture medium containing a basic FGF (fibroblast growth factor) that is known to have the property of maintaining the undifferentiated state of human iPS cells, and the like. For example, it is possible to use a human iPS cell medium (DMEM/Ham's F12 (Wako) containing 20% KnockOut serum replacement (KSR; Gibco), 1× non-essential amino acids (NEAA; Wako), 55 μmol/L 2-mercaptoethanol (2-ME; Gibco), 7.5 ng/mL recombinant human fibroblast growth factor 2 (FGF 2; PeproTech) and 0.5× penicillin and streptomycin (PS; Wako)), or Essential 8 medium (Thermo Fisher Scientific), STEMPRO (registered trademark) hESC SFM (Life Technologies Japan Ltd.), mTeSR1 (Veritas Corporation), TeSR2 (Veritas Corporation), StemFit (registered trademark), or the like, but there is no particular limitation.

The pluripotent stem cells may be maintenance-cultured on suitable feeder cells (for example, SL10 feeder cells, SNL feeder cells or the like) using an undifferentiated-state maintenance medium as mentioned above. Additionally, the pluripotent stem cells may be maintenance-cultured using the above-mentioned undifferentiated-state maintenance medium on cell culture dishes coated with a cell adhesion protein or an extracellular matrix such as vitronectin, fibronectin, laminin, collagen or matrigel.

The culture temperature is not particularly limited as long as it is a culture temperature suitable for culturing the pluripotent stem cells that are used. Generally, the temperature is 30° C. to 40° C., and is preferably about 37° C.

The cells are preferably cultured by using a CO₂ incubator or the like in an atmosphere with a CO₂ concentration of about 1% to 10%, preferably 5%.

The maintenance culture of the pluripotent stem cells may be maintained for a desired period of time by subculturing the cells, and it is preferable to form aggregates and induce differentiation by using the pluripotent stem cells, for example, 1 to 100 passages, preferably 10 to 50 passages, more preferably 25 to 40 passages after the maintenance culture.

### [6] Formation of Aggregates by Suspension Culturing Pluripotent Stem Cells

As one of the embodiments for forming an aggregate of pluripotent stem cells, cells that have been maintenance-cultured in the undifferentiated state may be detached from feeder cells by using accumax (Innovative Cell Technologies, Inc.) or the like, and the feeder cells are removed by rinsing three or four times with a human iPS cell culture medium. Next, the cells are broken up by pipetting into smaller cell clumps or single cells. Then the cells are suspended in a culture medium, and thereafter suspension cultured while stirring or rotating until the pluripotent stem cells in the suspension form aggregates. Preferable forms of the suspension culture are the same as the forms used when inducing the differentiation of the endodermal cells to primitive gut tube (PGT) cells in a suspension culture.

The culture temperature is not particularly limited as long as it is a culture temperature suitable for culturing the pluripotent stem cells that are used. Generally, the temperature is 30° C. to 40° C., and is preferably about 37° C.

The cells are preferably cultured by using a CO₂ incubator or the like in an atmosphere with a CO₂ concentration of about 1% to 10%, preferably 5%.

### [7] Preculturing of Pluripotent Stem Cells

Before inducing the differentiation of the above-mentioned pluripotent stem cell aggregates or pluripotent stem cells into endodermal cells, they may be suspension cultured by using a culture medium containing 2-mercaptoethanol to prepare a cell population.

The culture medium used in the preculture may, in accordance with the type of cells, be an MEM medium, a BME medium, a DMEM medium, a DMEM/F12 medium, an αMEM medium, an IMDM medium, an ES medium, a DM-160 medium, a Fisher medium, an F12 medium, a WE medium, an RPMI1640 medium, an Essential 6™ medium (Thermo Fisher Scientific), or the like.

The pluripotent stem cells are precultured in a suspension culture. The above-mentioned suspension culture conditions may be used, and furthermore, the cells may be suspension cultured by being adhered to a microcarrier or the like in advance, suspension cultured in the form of cell clumps composed only of cells, or a polymer such as collagen may be intermixed into the cell clumps. Thus, the form of the preculture is not particularly limited.

The concentration of 2-mercaptoethanol in the culture medium used for the preculture is not particularly limited as long as it is within a range in which the differentiation induction efficiency increases. For example, the concentration of 2-mercaptoethanol is preferably 1 μM or more, 2 μM or more, 5 μM or more, 10 μM or more, 20 μM or more, 30 μM or more, 40 μM or more, or 50 μM or more, and preferably 200 μM or less, 150 μM or less, 120 μM or less, 100 μM or less, 90 μM or less, 80 μM or less, 70 μM or less, or 60 μM or less.

The culture medium used for the preculture should also preferably be a culture medium to which FGF2 (fibroblast growth factor 2) is not added. In some cases, the efficiency of differentiation to endodermal cells can be increased by using a culture medium to which FGF2 is not added.

The culture medium used for the preculture should also preferably be a culture medium to which TGFβ1 (transforming growth factor (31) is not added. In some cases, the efficiency of differentiation to endodermal cells can be increased by using a culture medium to which TGFβ1 is not added.

The culture medium used for the preculture should also preferably be a culture medium to which a WNT signaling activator is not added. In some cases, the efficiency of differentiation to endodermal cells can be increased by using a culture medium to which a WNT signaling activator is not added.

The culture medium used for the preculture should also preferably be a culture medium to which activin A is not added. In some cases, the efficiency of differentiation to endodermal cells can be increased by using a culture medium to which activin A is not added.

Amino acids, antibiotics, antioxidants, and other additives may also be added to the culture medium used for the preculture. For example, it is possible to add 0.1% to 2% (volume/volume) of NEAA (non-essential amino acids), 0.1% to 2% (volume/volume) of penicillin/streptomycin, 0.1 to 20 mg/mL of BSA or 1% to 25% (volume/volume) (preferably 1% to 20% (volume/volume)) of KnockOut serum replacement (KSR), or the like.

The culture temperature is not particularly limited as long as it is a culture temperature suitable for culturing the pluripotent stem cells that are used. Generally, the temperature is 30° C. to 40° C., and is preferably about 37° C.

The cells are preferably cultured by using a CO₂ incubator or the like in an atmosphere with a CO₂ concentration of about 1% to 10%, preferably 5%.

The culture period of the preculture of pluripotent stem cells is not particularly limited as long as it is a number of days allowing the cells to be cultured until the pluripotency is increased. For example, it is sufficient that the period not exceed 1 week. More specifically, the culture period may be shorter than 6 days, shorter than 5 days, shorter than 4 days, shorter than 3 days, or 6 hours to 48 hours, about 12 hours to 36 hours, or 18 hours to 24 hours.

[8] Induced Differentiation into Endodermal Cells

In the present invention, the cell population obtained by the above-described preculture is cultured under conditions that allow induced differentiation to endodermal cells, thereby producing endodermal cells.

Endodermal cells have the ability to differentiate into the tissues of organs such as the digestive tract, the lung, the thyroid gland, the pancreas, and the liver, the cells of secretory glands opening onto the digestive tract, and the peritoneum, the pleura, the larynx, the auditory tube, the trachea, the bronchi, and the urinary tract (most of the bladder and the urethra, and part of the ureter). In general, they are sometimes referred to as the definitive endoderm (DE). Differentiation from pluripotent stem cells to endodermal cells can be confirmed by measuring the expression levels of genes specific to endodermal cells. Examples of genes specific to endodermal cells include SOX17, FOXA2, CXCR4, AFP, GATA4, EOMES, and the like. In the present description, endodermal cells are sometimes referred to alternatively as the definitive endoderm.

When inducing the pluripotent stem cells to differentiate into endodermal cells, the pluripotent stem cells are cultured by using a differentiation induction medium.

The differentiation induction medium is not particularly limited as long as it is a culture medium that induces the differentiation of pluripotent stem cells. Examples thereof include serum-containing media and serum-free media containing serum replacement components.

In accordance with the type of cells being used, it is possible to use a primate ES/iPS cell culture medium (ReproCELL medium), a BME medium, a BGJb medium, a CMRL 1066 medium, a Glasgow MEM medium, an Improved MEM Zinc Option medium, an IMDM medium, a Medium 199 medium, an Eagle MEM medium, an αMEM medium, a DMEM medium, a Ham's medium, an RPMI1640 medium, a Fischer's medium, and culture media obtained by mixing two or more media arbitrarily selected from these media. The culture medium is not particularly limited as long as it is a culture medium that can be used to culture animal cells.

The differentiation induction medium may contain a serum component or a serum replacement component. Examples of the serum component or the serum replacement component include albumin, insulin, transferrin, fatty acids, collagen precursors, trace elements (for example, zinc or selenium), B-27 Supplement (Thermo Fisher Scientific), N2 Supplement, N21 Supplement (R&D Systems), NeuroBrew-21 supplement (Miltenyi Biotec), KnockOut serum replacement (KSR), 2-mercaptoethanol, 3′thiolglycerol, and equivalents thereof.

Various additives, antibiotics, antioxidants, and the like may be further added to the differentiation induction medium. For example, it is possible to add 0.1 mM to 5 mM of sodium pyruvate, 0.1% to 2% (volume/volume) of non-essential amino acids, 0.1% to 2% (volume/volume) of penicillin, 0.1% to 2% (volume/volume) of streptomycin, and 0.1% to 2% (volume/volume) of amphotericin B, catalase, glutathione, galactose, retinoic acid (vitamin A), superoxide dismutase, ascorbic acid (vitamin C), D-α-tocopherol (vitamin E), and the like.

A differentiation-inducing factor is further added to the differentiation induction medium. Details regarding the differentiation-inducing factor will be described below.

The pluripotent stem cells are preferably cultured in a suspension culture during the induced differentiation. The cells may be suspension cultured by being adhered to a microcarrier or the like, suspension cultured in the form of cell clumps composed only of cells, or a polymer such as collagen may be intermixed into the cell clumps. Thus, the form of the culture is not particularly limited.

The culture temperature used when culturing the cells to induce differentiation is not particularly limited as long as it is a culture temperature suitable for culturing the pluripotent stem cells that are used. Generally, the temperature is 30° C. to 40° C., and is preferably about 37° C.

The cells are preferably cultured by using a CO₂ incubator or the like in an atmosphere with a CO₂ concentration of about 1% to 10%, preferably 5%.

The culture period for the differentiation culture from the pluripotent stem cells to endodermal cells is not particularly limited as long as the cells are converted to a cell type in which the cell properties of endodermal cells are exhibited. For example, it is sufficient for the period to be within 2 weeks. More specifically, the culture period may be 2 days or longer and 8 days or shorter, more preferably 2 days or longer and 7 days or shorter, and even more preferably 3 days or longer and 6 days or shorter. As an example, the culture period may be 4 or 5 days.

[9] Differentiation-Inducing Factor Used to Induce Differentiation into Endodermal Cells, and Other Additives

Preferably, the endodermal cells are endodermal cells that have been induced to differentiate by culturing a pluripotent stem cell population in a culture medium containing a TGFβ (transforming growth factor (3) superfamily signaling activator, and thereafter culturing the cells in a culture medium to which FGF2 and BMP4 (bone morphogenetic protein 4) are not added.

When activin A is used in the culture medium containing a TGFβ superfamily signaling activator, the initial concentration of activin A added is preferably 1 ng/mL or more, 2 ng/mL or more, 3 ng/mL or more, 5 ng/mL or more, 10 ng/mL or more, 20 ng/mL or more, 30 ng/mL or more, 40 ng/mL or more, or 50 ng/mL or more, and preferably 1,000 ng/mL or less, 900 ng/mL or less, 800 ng/mL or less, 700 ng/mL or less, 600 ng/mL or less, 500 ng/mL or less, 400 ng/mL or less, 300 ng/mL or less, 200 ng/mL or less, 150 ng/mL or less, or 100 ng/mL or less.

When FGF2 is used in the culture medium containing a TGFβ superfamily signaling activator, the initial concentration of FGF2 added is preferably 1 ng/mL or more, 2 ng/mL or more, 3 ng/mL or more, 5 ng/mL or more, 10 ng/mL or more, 20 ng/mL or more, 30 ng/mL or more, or 40 ng/mL or more, and preferably 1,000 ng/mL or less, 900 ng/mL or less, 800 ng/mL or less, 700 ng/mL or less, 600 ng/mL or less, 500 ng/mL or less, 400 ng/mL or less, 300 ng/mL or less, 200 ng/mL or less, 150 ng/mL, 100 ng/mL or less, 90 ng/mL or less, 80 ng/mL or less, or 70 ng/mL or less.

When BMP4 is used in the culture medium containing a TGFβ superfamily signaling activator, the initial concentration of BMP4 added is preferably 1 ng/mL or more, 2 ng/mL or more, 3 ng/mL or more, 5 ng/mL or more, 6 ng/mL or more, 7 ng/mL or more, 8 ng/mL or more, 9 ng/mL or more, 10 ng/mL or more, 11 ng/mL or more, 12 ng/mL or more, 13 ng/mL or more, 14 ng/mL or more, or 15 ng/mL or more, and preferably 1,000 ng/mL or less, 900 ng/mL or less, 800 ng/mL or less, 700 ng/mL or less, 600 ng/mL or less, 500 ng/mL or less, 400 ng/mL or less, 300 ng/mL or less, 200 ng/mL or less, 150 ng/mL, 100 ng/mL or less, 90 ng/mL or less, 80 ng/mL or less, 70 ng/mL or less, 60 ng/mL or less, 50 ng/mL or less, 40 ng/mL or less, or 30 ng/mL or less.

The culture medium to which FGF2 and BMP4 are not added preferably contains activin A.

When the culture medium to which FGF2 and BMP4 are not added contains activin A, the initial concentration of activin A added is preferably 1 ng/mL or more, 2 ng/mL or more, 3 ng/mL or more, 5 ng/mL or more, 10 ng/mL or more, 20 ng/mL or more, 30 ng/mL or more, 40 ng/mL or more, or 50 ng/mL or more, and preferably 1,000 ng/mL or less, 900 ng/mL or less, 800 ng/mL or less, 700 ng/mL or less, 600 ng/mL or less, 500 ng/mL or less, 400 ng/mL or less, 300 ng/mL or less, 200 ng/mL or less, 150 ng/mL or less, or 100 ng/mL or less.

The culture medium to which FGF2 and BMP4 are not added preferably contains at least one or more substances selected from the group consisting of insulin, transferrin, sodium selenite, and ethanolamine.

The concentration of insulin added is preferably 0.001 μg/mL or more, 0.01 μg/mL or more, 0.05 μg/mL or more, 0.1 μg/mL or more, or 0.2 μg/mL or more, and preferably 10,000 μg/mL or less, 1,000 μg/mL or less, 100 μg/mL or less, 10 μg/mL or less, 9 μg/mL or less, 8 μg/mL or less, 7 μg/mL or less, 6 μg/mL or less, 5 μg/mL or less, 4 μg/mL or less, 3 μg/mL or less, or 2 μg/mL or less. The concentration of transferrin added is preferably 0.001 μg/mL or more, 0.01 μg/mL or more, 0.05 μg/mL or more, 0.06 μg/mL or more, 0.07 μg/mL or more, 0.08 μg/mL or more, 0.09 μg/mL or more, 0.1 μg/mL or more, or 0.11 μg/mL or more, and preferably 10,000 μg/mL or less, 1,000 μg/mL or less, 100 μg/mL or less, 10 μg/mL or less, 9 μg/mL or less, 8 μg/mL or less, 7 μg/mL or less, 6 μg/mL or less, 5 μg/mL or less, 4 μg/mL or less, 3 μg/mL or less, 2 μg/mL or less, 1.9 μg/mL or less, 1.8 μg/mL or less, 1.7 μg/mL or less, 1.6 μg/mL or less, 1.5 μg/mL or less, 1.4 μg/mL or less, 1.3 μg/mL or less, 1.2 μg/mL or less, or 1.1 μg/mL or less. The concentration of sodium selenite added is preferably 0.001 ng/mL or more, 0.01 ng/mL or more, or 0.1 ng/mL or more, and preferably 10,000 ng/mL or less, 1,000 ng/mL or less, 100 ng/mL or less, 10 ng/mL or less, or 1 ng/mL or less. The concentration of ethanolamine added is preferably 0.001 μg/mL or more, 0.01 μg/mL or more, 0.02 μg/mL or more, 0.03 μg/mL or more, or 0.04 μg/mL or more, and preferably 10,000 μg/mL or less, 1,000 μg/mL or less, 100 μg/mL or less, 10 μg/mL or less, 1 μg/mL or less, 0.9 μg/mL or less, 0.8 μg/mL or less, 0.7 μg/mL or less, 0.6 μg/mL or less, 0.5 μg/mL or less, or 0.4 μg/mL or less.

It is preferable for the culture medium containing a TGFβ superfamily signaling activator and/or the culture medium to which FGF2 and BMP4 are not added to further contain 2-mercaptoethanol. The action of 2-mercaptoethanol can raise the efficiency of induced differentiation to endodermal cells.

It is preferable for the culture medium containing a TGFβ superfamily signaling activator to further contain a WNT signaling activator.

When CHIR99021 is used in the culture medium containing a TGFβ superfamily signaling activator, the initial concentration added is preferably 0.01 μM or more, 0.02 μM or more, 0.03 μM or more, 0.04 μM or more, 0.05 μM or more, 0.1 μM or more, 0.2 μM or more, 0.3 μM or more, 0.4 μM or more, 0.5 μM or more, 0.6 μM or more, 0.7 μM or more, 0.8 μM or more, 0.9 μM or more, 1 μM or more, or 2 μM or more, and preferably 100 μM or less, 90 μM or less, 80 μM or less, 70 μM or less, 60 μM or less, 50 μM or less, 45 μM or less, 40 μM or less, 35 μM or less, 30 μM or less, 25 μM or less, 20 μM or less, 15 μM or less, 10 μM or less, or 5 μM or less. More preferably, the initial concentration is 3 μM or 4 μM.

The culture medium containing a TGFβ superfamily signaling activator and/or the culture medium to which FGF2 and BMP4 are not added contains at least glucose. The lower limit of the concentration of glucose contained in the culture medium is not particularly limited as long as it is a concentration at which the cells can proliferate, but it should preferably be 0.01 g/L or more. Additionally, the upper limit of the concentration of glucose contained in the culture medium is not particularly limited as long as it is a concentration at which the cells do not die, but it should preferably be, for example, 10 g/L or less. As another embodiment, a culture medium containing less than 2.0 g/L of glucose is preferable for the purposes of achieving efficient differentiation to endodermal somatic cells. The glucose concentration in the culture medium containing a TGFβ superfamily signaling activator and/or the culture medium to which FGF2 and BMP4 are not added may be 1.0 g/L or less, 0.9 g/L or less, 0.8 g/L or less, 0.7 g/L or less, or 0.6 g/L or less. The lower limit of the glucose concentration in the case in which the culture medium containing a TGFβ superfamily signaling activator and/or the culture medium to which FGF2 and BMP4 are not added contains glucose is not particularly limited, and may be 0.01 g/L or more, 0.02 g/L or more, 0.05 g/L or more, 0.1 g/L or more, 0.2 g/L or more, 0.3 g/L or more, 0.4 g/L or more, or 0.5 g/L or more.

Hereinafter, the present invention will be explained in detail by providing examples, but the present invention is not limited to these examples.

### EXAMPLES

### Example 1

### <Maintenance Culture of Pluripotent Stem Cells>

The human iPS cell line TKDN4-M (The Institute of Medical Science, The University of Tokyo) was subjected to an undifferentiated-state maintenance culture using a human iPS cell medium (DMEM/Ham's F12 (Wako) containing 20% KnockOut serum replacement (KSR; Gibco), 1× non-essential amino acids (NEAA; Wako), 55 μmoL/L 2-mercaptoethanol (2-ME; Gibco), 7.5 ng/mL recombinant human fibroblast growth factor (FGF2; PeproTech), and 0.5× penicillin and streptomycin (PS; Wako)) on SNL feeder cells treated with mitomycin-C (Wako). Alternatively, the cell line was subjected to an undifferentiated-state maintenance culture on a plate coated with vitronectin (Gibco) using an Essential 8 medium (E8; Gibco) containing 1× penicillin, streptomycin and amphotericin B (Wako). The cells were cultured by adding Y27632 in such a way that the final concentration was 10 μM only at the time of seeding. The cells were cultured at 37° C. in an atmosphere with a 5% CO₂ concentration.

### <Preparation of Aggregates>

The human iPS cell line TkDN4-M (The Institute of Medical Science, The University of Tokyo) was rinsed once with PBS and incubated using accumax (Innovative Cell Technologies, Inc.) at 37° C. for 5 to 15 minutes, then dispersed to single cells by pipetting and collected. The cells, numbering 3×10⁷, were suspended in 30 mL of an mTeSR1 medium containing 10 μM of Y-27632, transferred to a 30 mL single-use bioreactor (ABLE Corporation), mounted on a six-channel magnetic stirrer (ABLE Corporation), and suspension cultured for 1 day in a 5% CO₂ incubator at 37° C., while stirring at a speed of 45 rpm.

### <Preculture of Pluripotent Stem Cells>

A cell population forming an aggregate obtained by the maintenance culture was suspended in DMEM/Ham's F12 (Wako) containing 20% (volume/volume) KnockOut serum replacement (KSR; Gibco), 1× non-essential amino acids (NEAA; Wako), 55 μmol/L 2-mercaptoethanol (Gibco), and 0.5× penicillin and streptomycin (PS; Wako), transferred to a 30 mL single-use bioreactor (ABLE Corporation), mounted on a six-channel magnetic stirrer (ABLE Corporation), and suspension cultured for 1 day in a 5% CO₂ incubator at 37° C., while stirring at a speed of 45 rpm.

### <Induced Differentiation to Endodermal Cells>

A cell population forming an aggregate obtained by the preculture was suspension cultured, on the first and second days, in RPMI1640 (Wako) containing 0.5% bovine serum albumin (BSA; Sigma), 0.4×PS, 1 mmol/L sodium pyruvate (Wako), 1×NEAA, 80 ng/mL recombinant human activin A (PeproTech), 50 ng/mL FGF2 (PeproTech), 20 ng/mL recombinant bone morphogenetic protein 4 (BMP4; PeproTech), and 3 μmol/L CHIR99021 (Wako). On the third day, BMP4, FGF2 and CHIR99021 were removed from this culture medium and the cells were suspension cultured, and on the fourth day, the cells were further suspension cultured for 1 day in a culture medium to which 1% KSR was added. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 45 rpm.

<Consideration of Method for Inducing Differentiation from Endodermal Cells to Primitive Gut Tube (PGT) Cells>

The endodermal cells obtained above were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 1% B27 supplement (Gibco), and 0.3% ITS-X (Wako). The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Reference Example 1

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), and 0.2 μM LDN193189 (Cayman). The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Reference Example 2

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 1% B27 supplement (Gibco), 0.67 μM EC23, 1 μM dorsomorphin, 10 μM SB431542, and 0.25 μM SANT1. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Example 2

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), 0.67 μM EC23, 10 μM SB431542, and 0.25 μM SANT1. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Comparative Example 1

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), 0.2 μM LDN193189 (Cayman), 10 μM SB431542, 0.25 μM SANT1, and 0.67 μM EC23. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Comparative Example 2

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), 0.2 μM LDN193189 (Cayman), 0.25 μM SANT1, and 0.67 μM EC23. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Comparative Example 3

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), 0.2 μM LDN193189 (Cayman), 10 μM SB431542, and 0.67 μM EC23. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Comparative Example 4

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech), 0.3% (V/V) ITS-X (Wako), 1% B27 supplement (Gibco), 0.2 μM LDN193189 (Cayman), 10 μM SB431542, and 0.25 μM SANT1. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### Comparative Example 5

<Consideration of Method for Inducing Differentiation from Endodermal Cells to Primitive Gut Tube (PGT) Cells>

Endodermal cells obtained by the same method as that in Example 1 were induced to differentiate to primitive gut tube (PGT) cells. Specifically, the cells were suspension cultured for 3 days in an RPMI1640 medium containing 0.25% BSA, 1 mmol/L sodium pyruvate, 1×NEAA, 0.4×PS, 50 ng/mL recombinant human FGF7 (PeproTech.), 0.3% (V/V) ITS-X (Wako), 1% B27 supplement (Gibco), 1 μM dorsomorphin, 10 μM SB431542, and 0.25 μM SANT1. The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 55 rpm.

### <Induced Differentiation to Pancreatic β Cells>

The primitive gut tube cells obtained by the methods described in Example 1, Reference Example 1, Reference Example 2, Example 2, Comparative Example 1, Comparative Example 2, Comparative Example 3, and Comparative Example 4 were induced to differentiate to pancreatic β cells in accordance with the method described in Yabe, S. G., Fukuda, S., Takeda, F., Nashiro, K., Shimoda, M., Okochi, H., “Efficient generation of functional pancreatic β-cells from human induced pluripotent stem cells”, J. Diabetes, 2017 February, 9(2):168-179.

Specifically, the differentiation to posterior foregut (PFG) cells was carried out by culturing the cells for 4 days in a DMEM medium (Wako) containing PS, NEAA, B27, EC23, and SANT1.

The differentiation to pancreatic progenitor (PP) cells was carried out by suspension culturing the cells for 3 days in a DMEM culture containing PS, NEAA, 50 ng/mL recombinant human FGF10 (PeproTech), B27, EC23, SANT1, Alk5 inhibitor II (BioVision), and indolactam V (ILV; Cayman).

The differentiation to endocrine progenitors was carried out by suspension culturing the cells for 3 to 7 days in a DMEM-based medium (Gibco) containing PS, B27, EC23, SANT1, Alk5 inhibitor II, and 50 ng/mL Exendin 4 (Sigma).

The differentiation to pancreatic β cells was carried out by suspension culturing the cells for 6 to 10 days in a DMEM-based medium containing PS, B27, 10 ng/mL of BMP4, 50 ng/mL of recombinant human hepatocyte growth factor (HGF; PeproTech), 50 ng/mL insulin-like growth factor 1 (IGF1; PeproTech), Alk5 inhibitor II, 50 ng/mL Exendin 4, 5 mmol/L of nicotinamide (Sigma), and 5 μmol/L forskolin (Wako). The cells obtained in this way are called iPS-β cells.

The suspension culture was implemented by mounting a 30 mL single-use bioreactor (ABLE Corporation) on a six-channel magnetic stirrer (ABLE Corporation), and suspension culturing the cells in a 5% CO₂ incubator at 37° C., while stirring at a speed of 65 rpm.

### [Analysis of Differentiation Efficiency]

Quantitative RT-PCR, according to the procedures indicated below, was used to investigate the differentiation efficiencies of the primitive gut tube cell populations produced in Example 1, Reference Example 1, Reference Example 2, Example 2, Comparative Example 1, Comparative Example 2, Comparative Example 3, and Comparative Example 4, and the differentiation efficiencies to iPS-β cells.

### <Quantitative RT-PCR>

The total RNA of the differentiation-induced primitive gut tube cells and iPS-β cells was isolated and purified using ISOGEN (Wako), and cDNA was synthesized using PrimeScript II (Takara Bio). Using the synthesized cDNA as a template, quantitative PCR was implemented by means of a MyiQ qPCR machine (Bio-Rad), using GoTaq qPCR master mix (Promega). The detection was performed by the intercalation method using SYBR Green, and the gene expression level comparison was carried out by the relative quantification method by means of the comparative Ct method. The expression level of each gene was standardized by OAZ1 or β-actin, which are housekeeping genes.

The base sequences of the primers used in the quantitative PCR are as indicated below.

HNF-1β F:
(SEQ ID NO: 1)
GAG ATC CTC CGA CAA TTC AAC C
HNF-1β R:
(SEQ ID NO: 2)
AAA CAG CAG CTG ATC CTG ACT G
HNF-4α F:
(SEQ ID NO: 3)
AAG AGA TCC ATG GTG TTC AAG GAC
HNF-4α R:
(SEQ ID NO: 4)
AGG TAG GCA TAC TCATTG TCA TCG
OAZ1 F:
(SEQ ID NO: 5)
GTC AGA GGG ATC ACA ATC TTT CAG
OAZ1 R:
(SEQ ID NO: 6)
GTC TTG TCG TTG GAC GTT AGT TC
INS F:
(SEQ ID NO: 7)
TTG TGA ACC AAC ACC TGT GC
INS R:
(SEQ ID NO: 8)
GTG TGT AGA AGA AGC CTC GTT CC
NKX6.1 F:
(SEQ ID NO: 9)
ATC TTC GCC CTG GAG AAG AC
NKX6.1 R:
(SEQ ID NO: 10)
CGT GCT TCT TCC TCC ACT TG
KIT F:
(SEQ ID NO: 11)
GCC ATC ATG GAG GAT GAC GA
KIT R:
(SEQ ID NO: 12)
TGC CAT CCA CTT CAC AGG TAG
RAP1A F:
(SEQ ID NO: 13)
GCC AAC AGT GTA TGC TCG AA
RAP1A R:
(SEQ ID NO: 14)
TCC GTG TCC TTA ACC CGT AA
FGF11 F:
(SEQ ID NO: 15)
GGC ATG ACT GAA CCT GCA TC
FGF11 R:
(SEQ ID NO: 16)
CGT ATG AGG TCT GGA GTG CAA
FGFR4 F:
(SEQ ID NO: 17)
TCC TTG ACC TCC AGC AAC GA
FGFR4 R:
(SEQ ID NO: 18)
GGC CTG TCC ATC CTT AAG CC
MDM2 F:
(SEQ ID NO: 19)
CCC GGA TTA GTG CGT ACG AG
MDM2 R:
(SEQ ID NO: 20)
GCA ATG GCT TTG GTC TAA CCA G
CASP3 F:
(SEQ ID NO: 21)
ACT GTG GCA TTG AGA CAG AC
CASP3 R:
(SEQ ID NO: 22)
TTT CGG TTA ACC CGG GTA AG
CDK1 F:
(SEQ ID NO: 23)
AGG TCA AGT GGT AGC CAT GA
CDK1 R:
(SEQ ID NO: 24)
TGT ACT GAC CAG GAG GGA TAG
β-Actin F:
(SEQ ID NO: 25)
CCT CAT GAA GAT CCT CAC CGA
β-Actin R:
(SEQ ID NO: 26)
TTG CCA ATG GTG ATG ACC TGG

### <Measurement Results>

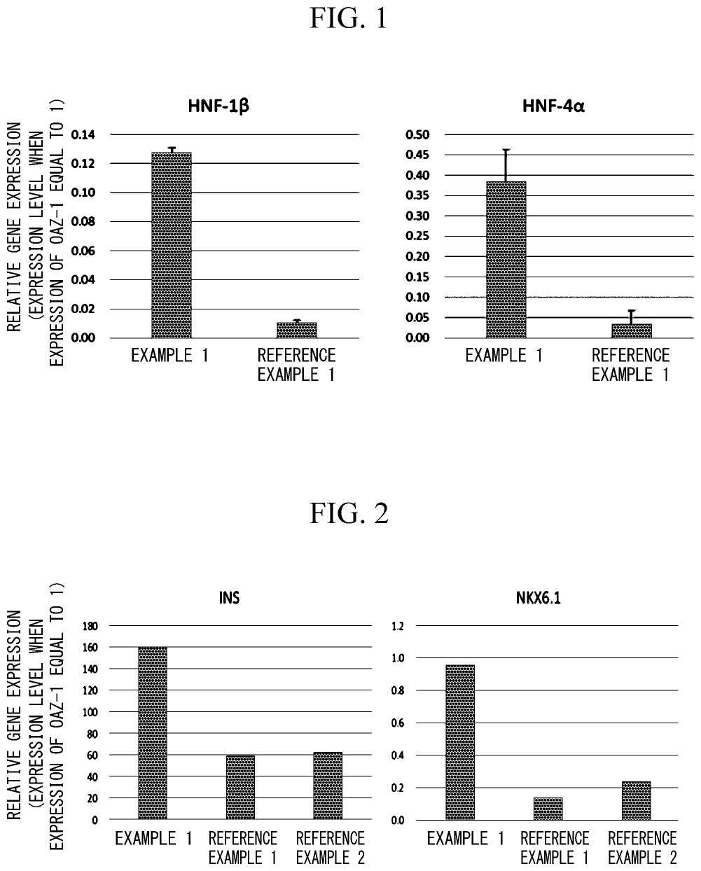

The results of the measurements of the gene expression levels are indicated in FIG. 1 and FIG. 2.

The results in FIG. 1 and FIG. 2 are summarized in the tables below.

TABLE 1

Relative expression level when
expression of OAZ-1 equal to 1

| | Mean | Standard Deviation |
|---|---|---|
| HNF-1β | | |
| Example 1 (n = 2) | 0.12730 | 0.00368 |
| Reference Example 1 (n = 2) | 0.01001 | 0.00195 |
| HNF-4α | | |
| Example 1 (n = 2) | 0.38316 | 0.07928 |
| Reference Example 1 (n = 2) | 0.03352 | 0.03259 |

TABLE 2

Relative expression level when
expression of OAZ-1 equal to 1

| | Mean | Standard Deviation |
|---|---|---|
| INS | | |
| Example 1 (n = 5) | 160.84 | 37.95 |
| Reference Example 1 (n = 4) | 59.64 | 10.34 |
| Reference Example 2 (n = 1) | 62.96 | — |
| NKX6.1 | | |
| Example 1 (n = 5) | 0.959 | 0.373 |
| Reference Example 1 (n = 4) | 0.135 | 0.027 |
| Reference Example 2 (n = 1) | 0.237 | — |

In both Example 1 and Reference Example 1, induced differentiation to primitive gut tube cells was observed. Additionally, the expression levels of the PGT marker (HNF-1β and HNF-4α) genes were elevated in the primitive gut tube cells obtained by the method described in Example 1 relative to the primitive gut tube cells obtained by the method described in Reference Example 1 (FIG. 1), thus demonstrating that the induced differentiation to primitive gut tube cells in Example 1 was higher than that in Reference Example 1. In other words, it was demonstrated that efficiency of induced differentiation to primitive gut tube cells was elevated when the endodermal cells were cultured in the absence of a bone morphogenetic protein (BMP) signaling inhibitor.

The expression of the INS gene and the NKX6.1 gene was elevated in cells obtained by inducing primitive gut tube cells obtained by the method described in Example 1 to further differentiate into iPS-β cells relative to cells obtained by inducing primitive gut tube cells obtained by the methods described in Reference Example 1 and Reference Example 2 to further differentiate into iPS-β cells (FIG. 2). Additionally, the expression of the INS gene was elevated in cells obtained by inducing primitive gut tube cells obtained by the method described in Example 2 to further differentiate into iPS-β cells relative to cells obtained by inducing primitive gut tube cells obtained by the methods described in Comparative Example 1 to Comparative Example 4 to further differentiate into iPS-β cells (FIG. 4).

Thus, it was demonstrated that the differentiation induction efficiency to primitive gut tube cells in Example 1 and Example 2 was higher than that in Reference Example 1 and Reference Example 2. In other words, it was demonstrated that the differentiation induction efficiency to primitive gut tube cells was elevated by culturing endodermal cells in the absence of a bone morphogenetic protein (BMP) signaling inhibitor. Additionally, the expression of the INS gene was reduced in cells obtained by inducing primitive gut tube cells obtained by the method described in Comparative Example 4 to further differentiate into iPS-β cells. Thus, the expression of the INS gene can be considered to be elevated by culturing endodermal cells in the absence of a BMP signaling inhibitor and by further adding a retinoic acid analog or the like (FIG. 4).

### Example 3

### <Transplantation Experiment on Diabetes Model Mice (Non-Obese Diabetic (NOD)-Severe Combined Immunodeficiency (SCID) Diabetes Model Mice Experiment)>

The iPS-β cells obtained by the induced differentiation of the primitive gut tube cells obtained by the methods in Example 1 and Reference Example 2 to pancreatic β cells were rinsed once with HBSS, thereafter suspended in HBSS containing 3.33 μg/mL iMatrix-511 (Wako), and the suspended cells were transplanted (6×10⁶ cells were administered) under the left renal capsules of NOD/SCID diabetes model mice (CLEA Japan, Inc.) using a Hamilton syringe (Hamilton Company). As the NOD/SCID diabetes model mice, individuals having blood glucose levels elevated to 250 mg/dL or more by administering 130 mg/kg of streptozotocin (STZ; Sigma) via the caudal vein were used. The transplantation (day 0) was implemented 14 days after administering STZ (−14 days). The casual blood glucose levels were measured by collecting blood from the caudal vein and using Glutest Neo alpha (Sanwa Kagaku).

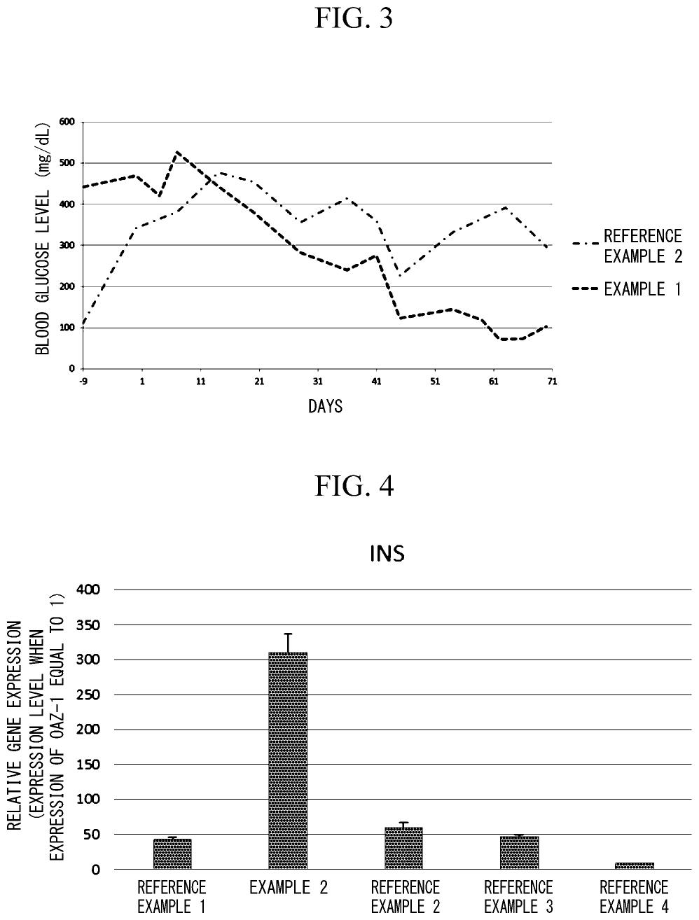

FIG. 3 shows the results of measurement of the casual blood glucose levels in the diabetes model mice.

In individual mice transplanted with iPS-β cells obtained by induced differentiation from the primitive gut tube cells prepared by the method in Example 1 (FIG. 3, Example 1), the blood glucose level fell to normal levels (200 mg/dL or lower) in about 40 days after transplantation. Thus, it can be understood that the iPS-β cells control blood glucose levels. In contrast therewith, in individual mice transplanted with iPS-β cells obtained by induced differentiation from the primitive gut tube cells prepared by the method in Reference Example 2 (FIG. 3, Reference Example 2), the blood glucose level did not fall to normal levels (200 mg/dL or lower) even when 71 days elapsed after transplantation. From these results, somatic cells (pancreatic β cells) obtained by induced differentiation of primitive gut tube cells obtained by the method of the present invention can be expected to be effective in applications to the treatment of diabetes and the like. Additionally, the primitive gut tube cells produced by the method of the present invention can be considered to be able to differentiate into pancreatic β cells that are optimal for use in cell therapy formulations.

### [Example 4] Comprehensive Gene Expression Analysis of Primitive Gut Tube Cell Populations

Comprehensive gene expression analyses of primitive gut tube cell populations were implemented by the method indicated below.

### <RNA Extraction>

The total RNA of endodermal cells induced to differentiate by being cultured by the method described in Example 1 and primitive gut tube cells induced to differentiate by being cultured by the methods described in Example 1, Reference Example 2 and Comparative Example 5 were isolated and purified using ISOGEN (Wako).

### <DNA Microarray Analysis>

The extracted total RNA were used to perform DNA microarray analysis.

(1) cRNA Synthesis

A 3′ IVT PLUS Reagent Kit was used to perform cRNA synthesis. The method followed the protocol recommended by Affymetrix (registered trademark). The total RNA (100 ng) were used to prepare cDNA by a reverse transcription reaction. The produced cDNA were transcribed to cRNA by means of in vitro transcription and the cRNA were biotin-labeled.

### (2) Hybridization

The labeled cRNA (12.5 μg) were added to a hybridization buffer and hybridized for 16 hours on a Human Genome U133 Plus 2.0 Array. After rinsing and phycoerythrin-dyeing the cRNA in a GeneChip (registered trademark) Fluidics Station 450, they were scanned with a GeneChip (registered trademark) Scanner 3000 7G, image-analyzed with AGCC (Affymetrix (registered trademark) GeneChip (registered trademark) Command Console (registered trademark) software), and quantified by using an Affymetrix (registered trademark) Expression Console™.

Regarding the steps in (1) and (2) above, a contractual analysis service using the Affymetrix DNA microarray “GeneChip (registered trademark) Human Genome U133 Plus 2.0 Array”, provided by Riken Genesis Co., Ltd. was utilized (https://rikengenesis.jp/contents/ja_JPY/microarray_affymetrix.html). This contractual analysis service is a service that performs comprehensive gene expression analysis using GeneChip (registered trademark) simply by being provided with samples (total RNA or the like).

### <Enrichment Analysis Using DNA Microarray Data>

Statistical analysis of the data was performed using R version 3.4.2 and Bioconductor version 3.6 (The R Foundation for Statistical Computing, 2017). Additionally, enrichment analysis was performed using the Database for Annotation, Visualization and Integrated Discovery (DAVID) 6.8 (National Institute of Allergy and Infectious Diseases (NIAID), NIH) (https://david.ncifcrf.gov/home.jsp).

The Affymetrix array data (CEL file) obtained by the above-mentioned DNA microarray analysis and Affymetrix array data (CEL file) from a primitive gut tube cell population induced to differentiate by being cultured by the method described in Reference Example 2 were read into R with a ReadAffy( ) function. Thereafter, an rma( ) function was used to normalize the microarray data. This rma( ) function is a function for implementing a robust multi-array average (RMA) method (Irizarry, R., Hobbs, B., Collin, F., Beazer-Barclay, Y, Antonellis, K., Scherf, U., Speed, T., “Exploration, normalization, and summaries of high density oligonucleotide array probe level data”, Biostatistics, 2003, 4:249). The RMA method is currently one of the normalization methods that are most commonly used, and is a method for performing background correction, normalization, and calculating expression at once. The RMA method involves performing a normalization procedure by applying a base-2 logarithmic transformation to a perfect match (PM) value. Thus, the normalized result is also output as a base-2 logarithmic transformation value.

Next, for the normalized signals, the difference between the signal value from the primitive gut tube cell population induced to differentiate by culturing cells with the method described in Example 1 and the signal value from the primitive gut tube cell population induced to differentiate by culturing cells with the method described in Reference Example 2 was calculated.

The normalized signals obtained by the RMA method have undergone a base-2 logarithmic transformation. Therefore, the difference between the signals is (log₂(x)−log₂(y)=log₂(x/y)), which is the base-2 logarithmic transformation value of the fold change. Thus, the fold change was calculated by performing an inverse transformation on the difference. Thereafter, transcription products having a fold change of 2 or higher and transcription products having a fold change of 0.5 or lower were extracted and respectively defined as differentially expressed genes. In this experiment, there is no repetition of samples at each condition and there is just one sample. Thus, it is not possible to select a differentially expressed gene by t-testing or related hypothesis testing. For this reason, as standards that are generally used, standards in which the fold change is 2 or higher and 0.5 or lower were employed. Transcription products in which the fold change was 2 or higher and 0.5 or lower were used as the targets for enrichment analysis.

Enrichment analysis is a method in which, among differentially expressed genes, those having many functions are analyzed, and is one of annotation analysis. For example, it is possible to analyze whether the differentially expressed genes include relatively more transcription factors, relatively more cell cycles or the like in terms of probability theory. The transcription product list selected in the above-mentioned analysis was read into DAVID without information such as expression levels, and enrichment analysis was performed. Although various types of enrichment analysis are possible with DAVID, the analysis was limited to KEGG pathway analysis on this occasion in order to reduce the risk of redundancy by implementing many analyses.

KEGG pathway analysis is performed by DAVID accessing the Kyoto Encyclopedia of Genes and Genomes (KEGG) database (www.genome.jp/kegg/) and statistically extracting pathways highly correlated with the transcription product list. Although p values regarding the correlations to the respective pathways are displayed, it is possible to compute various redundancy-adjusted p values with DAVID because hypothesis testing is implemented on multiple pathways at once. In the current analysis, the Benjamini-Hochberg method, which is the most commonly used in microarray analysis, was used (Benjamini, Y., Hochberg, Y, 1995, “Controlling the false discovery rate: a practical and powerful approach to multiple testing”, Journal of the Royal Statistical Society, Series B, 57(1): 289-300). The adjusted p values computed by the Benjamini-Hochberg method were used to select pathways with high correlations to the signal set. The Bonferroni method was used to further adjust for redundancies, and when the adjusted p value was less than 0.05/20>0.0033=3.3×10⁻³, that pathway was determined as being a statistically significant pathway.

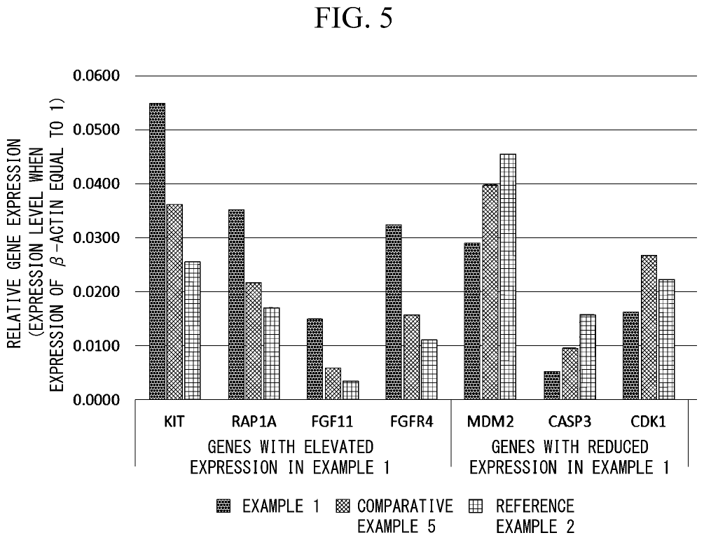

As a result of the enrichment analysis, the gene expression on the pathway “Biosynthesis of amino acids” (http://www.genome.jp/kegg-bin/show_pathway?map=hsa04015&show_description=show), the pathway “Rap1 signaling pathway” (http://www.genomejp/kegg-bin/show_pathway?map=hsa04015&show_description=show), and the pathway “Pathways in cancer” (http://www.genome.jp/kegg-bin/show_pathway?map=hsa05200&show_description=show) were elevated, and the gene expression on the pathway “p53 signaling pathway” (http://www.genome.jp/kegg-bin/show_pathway?map=hsa04115&show_description=show) was reduced in the primitive gut tube cell population induced to differentiate by being cultured by the method described in Example 1 in comparison with the primitive gut tube cell population induced to differentiate by being cultured by the method described in Reference Example 2. The genes that were differentially expressed are shown in Tables 3 to 6. When expression analysis by quantitative RT-PCR was performed by the above-mentioned method for the genes included above, for example, expression was elevated in Example 1 in comparison with Reference Example 2 or Comparative Example 5 for the KIT gene, the RAP1A gene, the FGF11 gene, and the FGFR4 gene, and expression was reduced in Example 1 in comparison with Reference Example 2 or Comparative Example 5 for the MDM2 gene, the CASP3 gene, and the CDK1 gene (FIG. 5). The numerical values in the graph shown in FIG. 5 are indicated in Table 7. Therefore, due to the changes in the expression levels of these genes obtained from the results of the enrichment analysis, the differentiation efficiency to primitive gut tube cells and iPS-β cells can be considered to be increased. The housekeeping gene in FIG. 5 and Table 7 is β-actin.

### <Extraction of Differentiated Marker Gene Using DNA Microarray Data>

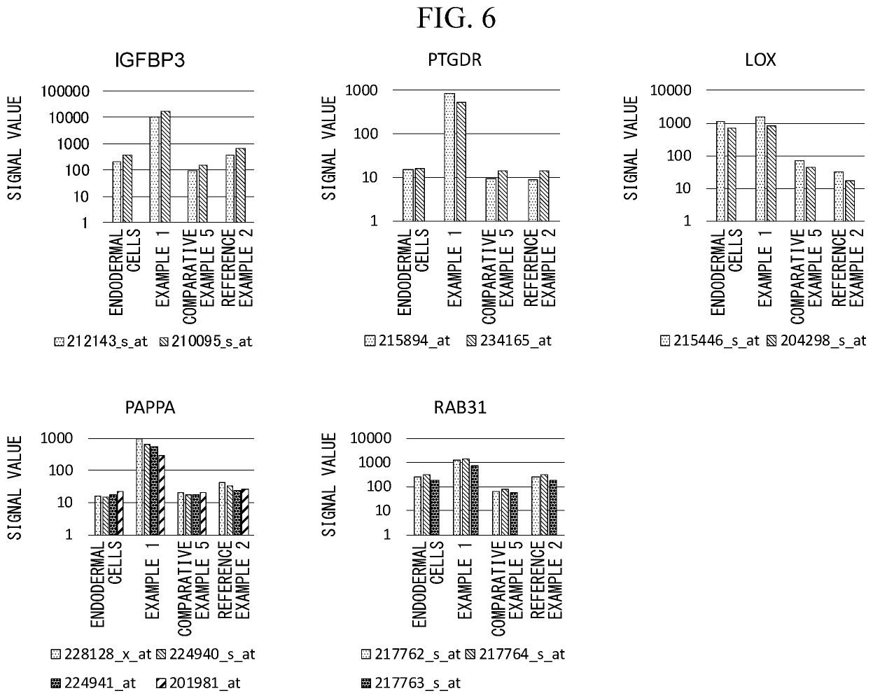

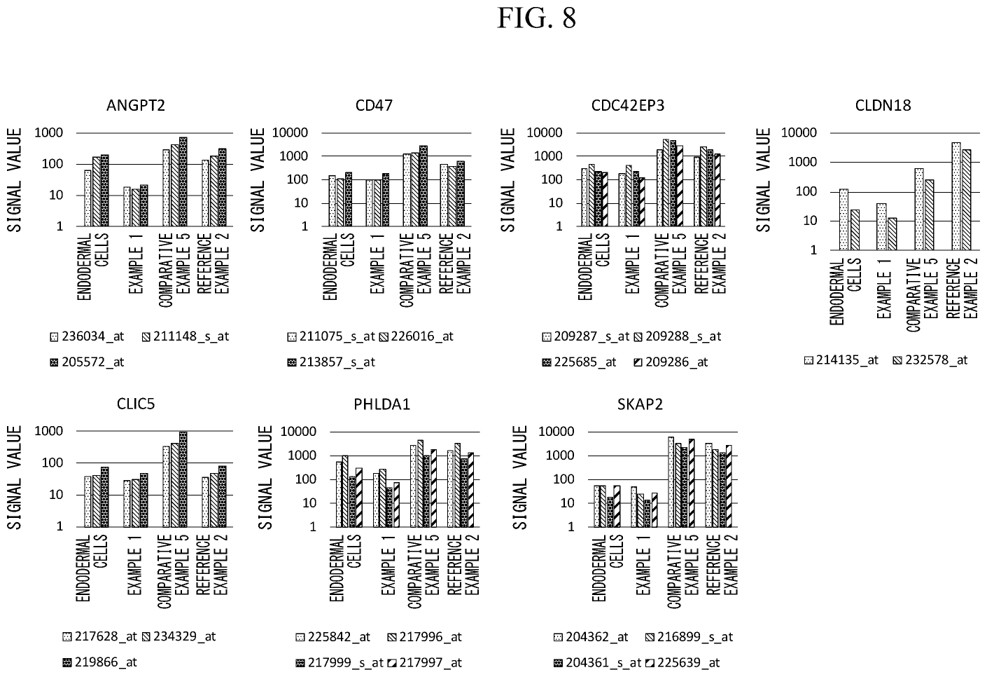

Data (CHP file) obtained by using Affymetrix (registered trademark) Expression Console™ software to convert the Affymetrix array data (CEL file) obtained by the above-mentioned DNA microarray analysis was read into Transcriptome Analysis Console™ software to obtain signal values (base-2 logarithmic transformation values). These signal values were converted to integers. Among the genes obtained from the primitive gut tube cell population cultured by the method described in Example 1, the genes in which the signal values of the genes (probes) were ten or more times or one-tenth or less in comparison with the signal values of the genes (probes) obtained from the primitive gut tube cell population cultured by the method described in Comparative Example 5 were extracted and are indicated in FIG. 6 and FIG. 8. The numerical values of the graphs shown in FIG. 6 are indicated in Table 8 and the numerical values of the graphs shown in FIG. 8 are indicated in Table 9. These genes can be considered to be potential gene markers suitable for use in a differentiation induction culture.

TABLE 3
Differentially expressed genes relating to
“biosynthesis of amino acids”

| Gene Name (abbreviation) | ENSEMBL_GENE_ID |
|---|---|
| N-acetylglutamate synthase (NAGS) | ENSG00000161653 |
| aldolase, fructose-bisphosphate A (ALDOA) | ENSG00000149925 |
| aldolase, fructose-bisphosphate C (ALDOC) | ENSG00000109107 |
| aminoadipate aminotransferase (AADAT) | ENSG00000109576 |
| argininosuccinate synthase 1 (ASS1) | ENSG00000130707 |
| branched chain amino acid transaminase 1 | ENSG00000060982 |
| (BCAT1) | |
| enolase 1 (ENO1) | ENSG00000074800 |
| enolase 2 (ENO2) | ENSG00000111674 |
| glutamate-ammonia ligase (GLUL) | ENSG00000135821 |
| phosphofructokinase, liver type (PFKL) | ENSG00000141959 |
| phosphofructokinase, platelet (PFKP) | ENSG00000067057 |
| phosphoglycerate kinase 1 (PGK1) | ENSG00000102144 |
| phosphoserine phosphatase (PSPH) | ENSG00000146733 |
| pyrroline-5-carboxylate reductase 1 (PYCR1) | ENSG00000183010 |
| pyruvate kinase, muscle (PKM) | ENSG00000067225 |
| serine hydroxymethyltransferase 2 (SHMT2) | ENSG00000182199 |
| transketolase (TKT) | ENSG00000163931 |

TABLE 4
Differentially expressed genes relating
to “Rap1 signaling pathway”

| Gene Name (abbreviation) | ENSEMBL_GENE_ID |
|---|---|
| KIT proto-oncogene receptor tyrosine kinase | ENSG00000157404 |
| (KIT) | |
| RAP1A, member of RAS oncogene family | ENSG00000116473 |
| (RAP1A) | |
| Rap guanine nucleotide exchange factor 4 | ENSG00000091428 |
| (RAPGEF4) | |
| adenylate cyclase 7 (ADCY7) | ENSG00000121281 |
| adenylate cyclase 8 (ADCY8) | ENSG00000155897 |
| afadin, adherens junction formation factor | ENSG00000130396 |
| (AFDN) | |
| amyloid beta precursor protein binding family | ENSG00000077420 |
| B member 1 interacting protein (APBB1IP) | |
| angiopoietin 1 (ANGPT1) | ENSG00000154188 |
| calmodulin 1 (CALM1) | ENSG00000198668 |
| ephrin A1 (EFNA1) | ENSG00000169242 |
| ephrin A3 (EFNA3) | ENSG00000143590 |
| ephrin A5 (EFNA5) | ENSG0000018434 |
| fibroblast growth factor 11 (FGF11) | ENSG00000161958 |
| fibroblast growth factor receptor 3 (FGFR3) | ENSG00000068078 |
| fibroblast growth factor receptor 4 (FGFR4) | ENSG00000160867 |
| glutamate ionotropic receptor NMDA type | ENSG00000183454 |
| subunit 2A (GRIN2A) | |
| insulin like growth factor 1 (IGF1) | ENSG00000017427 |
| membrane associated guanylate kinase, WW | ENSG00000081026 |
| and PDZ domain containing 3 (MAGI3) | |
| phosphoinositide-3-kinase regulatory subunit 1 | ENSG00000145675 |
| (PIK3R1) | |
| phosphoinositide-3-kinase regulatory subunit 5 | ENSG00000141506 |
| (PIK3R5) | |
| phospholipase C beta 1 (PLCB1) | ENSG00000182621 |
| phospholipase C epsilon 1 (PLCE1) | ENSG00000138193 |
| placental growth factor (PGF) | ENSG00000119630 |
| platelet derived growth factor D (PDGFD) | ENSG00000170962 |
| platelet derived growth factor receptor alpha | ENSG00000134853 |
| (PDGFRA) | |
| regulator of G-protein signaling 14 (RGS14) | ENSG00000169220 |
| signal induced proliferation associated 1 like 2 | ENSG00000116991 |
| (SIPA1L2) | |
| talin 2 (TLN2) | ENSG00000171914 |
| vascular endothelial growth factor C (VEGFC) | ENSG00000150630 |

TABLE 5
Differentially expressed genes relating to “Pathways in cancer”

| Gene Name (abbreviation) | ENSEMBL_GENE_ID |
|---|---|
| A-Raf proto-oncogene, serine/threonine kinase | ENSG00000078061 |
| (ARAF) | |
| BCR, RhoGEF and GTPase activating protein | ENSG00000186716 |
| (BCR) | |
| C-X-C motif chemokine receptor 4 (CXCR4) | ENSG0000012196 |
| CCAAT/enhancer binding protein alpha | ENSG00000245848 |
| (CEBPA) | |
| Cbl proto-oncogene C (CBLC) | ENSG00000142273 |
| KIT proto-oncogene receptor tyrosine kinase | ENSG00000157404 |
| (KIT) | |
| MDS1 and EVI1 complex locus (MECOM) | ENSG00000085276 |
| SMAD family member 3 (SMAD3) | ENSG00000166949 |
| adenylate cyclase 7 (ADCY7) | ENSG00000121281 |
| adenylate cyclase 8 (ADCY8) | ENSG00000155897 |
| catenin alpha 3 (CTNNA3) | ENSG00000183230 |
| collagen type IV alpha 3 chain (COL4A3) | ENSG00000169031 |
| collagen type IV alpha 5 chain (COL4A5) | ENSG00000188153 |
| collagen type IV alpha 6 chain (COL4A6) | ENSG00000197565 |
| cyclin D1 (CCND1) | ENSG00000110092 |
| egl-9 family hypoxia inducible factor 1 | ENSG0000013576 |
| (EGLN1) | |
| endothelial PAS domain protein 1 (EPAS1) | ENSG00000116016 |
| endothelin receptor type A (EDNRA) | ENSG00000151617 |
| fibronectin 1 (FN1) | ENSG00000115414 |
| frizzled class receptor 1 (FZD1) | ENSG00000157240 |
| frizzled class receptor 2 (FZD2) | ENSG00000180340 |
| laminin subunit beta 1 (LAMB1) | ENSG00000091136 |
| lysophosphatidic acid receptor 6 (LPAR6) | ENSG00000139679 |
| mitogen-activated protein kinase 10 (MAPK10) | ENSG00000109339 |
| patched 1(PTCH1) | ENSG00000185920 |
| peroxisome proliferator activated receptor | ENSG00000132170 |
| gamma (PPARG) | |
| phosphoinositide-3-kinase regulatory subunit | ENSG00000145675 |
| 1 (PIK3R1) | |
| phosphoinositide-3-kinase regulatory subunit | ENSG00000141506 |
| 5(PIK3R5) | |
| phospholipase C beta 1 (PLCB1) | ENSG00000182621 |
| phospholipase C gamma 2 (PLCG2) | ENSG00000197943 |
| prostaglandin E receptor 2 (PTGER2) | ENSG00000125384 |
| protein inhibitor of activated STAT 2 (PIAS2) | ENSG00000078043 |
| retinoid X receptor alpha (RXRA) | ENSG00000186350 |
| solute carrier family 2 member 1 (SLC2A1) | ENSG00000117394 |
| transforming growth factor beta 1 (TGFB1) | ENSG00000105329 |
| transforming growth factor beta receptor 2 | ENSG00000163513 |
| (TGFBR2) | |
| tropomyosin 3 (TPM3) | ENSG00000143549 |
| vascular endothelial growth factor C (VEGFC) | ENSG00000150630 |

TABLE 6
Differentially expressed genes relating to “p53 signaling pathway”

| Gene Name (abbreviation) | ENSEMBL_GENE_ID |
|---|---|
| BCL2 associated X, apoptosis regulator (BAX) | ENSG00000087088 |
| Fas cell surface death receptor (FAS) | ENSG00000026103 |
| MDM2 proto-oncogene (MDM2) | ENSG00000135679 |
| PERP, TP53 apoptosis effector (PERP) | ENSG00000112378 |
| STEAP3 metalloreductase (STEAP3) | ENSG00000115107 |
| caspase 3 (CASP3) | ENSG00000164305 |
| caspase 8 (CASP8) | ENSG00000064012 |
| cyclin D2 (CCND2) | ENSG00000118971 |
| cyclin E2 (CCNE2) | ENSG00000175305 |
| cyclin dependent kinase 1 (CDK1) | ENSG00000170312 |
| cyclin dependent kinase 6 (CDK6) | ENSG00000105810 |
| cyclin dependent kinase inhibitor 1A | ENSG00000124762 |
| (CDKN1A) | |
| damage specific DNA binding protein 2 | ENSG00000134574 |
| (DDB2) | |
| phorbol-12-myristate-13-acetate-induced | ENSG00000141682 |
| protein 1 (PMAIP1) | |
| protein phosphatase, Mg2+/Mn2+ dependent | ENSG00000170836 |
| 1D (PPM1D) | |
| reprimo, TP53 dependent G2 arrest mediator | ENSG00000177519 |
| candidate (RPRM) | |
| ribonucleotide reductase regulatory TP53 | ENSG00000048392 |
| inducible subunit M2B (RRM2B) | |
| serpin family B member 5 (SERPINB5) | ENSG00000206075 |
| serpin family E member 1 (SERPINE1) | ENSG00000106366 |
| sestrin 2 (SESN2) | ENSG00000130766 |
| stratifin (SFN) | ENSG00000175793 |
| zinc finger matrin-type 3 (ZMAT3) | ENSG00000172667 |

TABLE 7
Relative expression level when expression of β-actin equal to 1

| | Genes with elevated | | | | Genes with reduced | | |
|---|---|---|---|---|---|---|---|
| | expression in Example 1 | | | | expression in Example 1 | | |
| | KIT | RAP1A | FGF11 | FGFR4 | MDM2 | CASP3 | CDK1 |
| Example 1 | 0.0548 | 0.0351 | 0.0150 | 0.0323 | 0.0290 | 0.0052 | 0.0162 |
| Comparative | 0.0361 | 0.0216 | 0.0058 | 0.0155 | 0.0397 | 0.0096 | 0.0267 |
| Example 5 | | | | | | | |
| Reference | 0.0255 | 0.0170 | 0.0034 | 0.0111 | 0.0454 | 0.0157 | 0.0222 |
| Example 2 | | | | | | | |

TABLE 8

| Gene | | | Endodermal | | Comparative | Reference |
|---|---|---|---|---|---|---|
| Symbol | Transcript ID | Probe ID | Cells | Example 1 | Example 5 | Example 2 |
| IGFBP3 | Hs.77326.1 | 212143_s_at | 206.50 | 10660.59 | 89.88 | 364.56 |
| | g183115 | 210095_s_at | 372.22 | 17079.76 | 150.12 | 689.78 |
| PTGDR | Hs.158326.0 | 215894_at | 14.72 | 855.13 | 9.19 | 9.06 |
| | Hs.306831.0 | 234165_at | 15.89 | 541.19 | 14.32 | 14.12 |
| LOX | Hs.102267.3 | 215446_s_at | 1160.07 | 1520.15 | 69.07 | 32.00 |
| | g4505008 | 204298_s_at | 719.08 | 792.35 | 43.11 | 17.27 |
| PAPPA | Hs.250655.5 | 228128_x_at | 16.00 | 942.27 | 21.11 | 42.81 |
| | Hs.250655.4 | 224940_s_at | 14.93 | 630.35 | 17.75 | 32.45 |
| | Hs.250655.4 | 224941_at | 17.27 | 533.74 | 17.39 | 23.75 |
| | Hs.75874.0 | 201981_at | 22.78 | 290.02 | 20.39 | 25.99 |
| RAB31 | Hs.223025.0 | 217762_s_at | 254.23 | 1296.13 | 62.68 | 259.57 |
| | g9963780 | 217764_s_at | 308.69 | 1458.23 | 77.17 | 315.17 |
| | g5803130 | 217763_s_at | 184.82 | 714.11 | 57.68 | 176.07 |

TABLE 9

| Gene | | | Endodermal | | Comparative | Reference |
|---|---|---|---|---|---|---|
| Symbol | Transcript ID | Probe ID | Cells | Example 1 | Example 5 | Example 2 |
| ANGPT2 | Hs.68301.0 | 236034_at | 63.12 | 19.29 | 292.04 | 138.14 |
| | g8570646 | 211148_s_at | 170.07 | 16.00 | 427.57 | 178.53 |
| | g4557314 | 205572_at | 198.09 | 21.71 | 709.18 | 306.55 |
| BMPR1B | Hs.72472.0 | 229975_at | 89.26 | 191.34 | 2665.15 | 190.02 |
| | g2055308 | 210523_at | 25.81 | 25.46 | 352.14 | 29.04 |
| | Hs.161712.0 | 242579_at | 28.64 | 79.89 | 1152.06 | 86.82 |
| CD47 | g396704 | 211075_s_at | 147.03 | 103.25 | 1305.15 | 477.71 |
| | Hs.76728.0 | 226016_at | 109.90 | 105.42 | 1398.83 | 374.81 |
| | Hs.82685.1 | 213857_s_at | 215.27 | 190.02 | 2759.13 | 648.07 |
| CDC42EP3 | g4324453 | 209287_s_at | 298.17 | 181.02 | 2005.85 | 968.76 |
| | g6807668 | 209288_s_at | 481.04 | 415.87 | 5518.27 | 2610.30 |
| | Hs.6774.0 | 225685_at | 227.54 | 216.77 | 5042.77 | 1964.57 |
| | Hs.260024.0 | 209286_at | 202.25 | 127.12 | 3019.30 | 1243.34 |
| CLDN18 | Hs.16762.0 | 214135_at | 126.24 | 41.07 | 643.59 | 4837.35 |
| | Hs.16762.1 | 232578_at | 24.42 | 12.47 | 247.28 | 2683.69 |
| CLIC5 | Hs.266784.0 | 217628_at | 38.59 | 28.05 | 326.29 | 36.25 |
| | Hs.283855.0 | 234329_at | 42.22 | 30.70 | 410.15 | 46.85 |
| | g8393146 | 219866_at | 77.17 | 48.84 | 929.30 | 83.87 |
| FRZB | g4503788 | 203698_s_at | 1618.00 | 177.29 | 5220.60 | 125.37 |
| | g1917006 | 203697_at | 2048.00 | 172.45 | 6382.92 | 163.14 |
| IGF2; INS-IGF2 | g6453816 | 202410_x_at | 27.47 | 27.67 | 292.04 | 32.67 |
| | g182527 | 210881_s_at | 24.08 | 28.05 | 306.55 | 32.22 |
| | Hs.251664.0 | 202409_at | 37.27 | 69.07 | 6427.31 | 235.57 |
| PHLDA1 | Hs.288850.0 | 225842_at | 526.39 | 184.82 | 2740.08 | 1629.26 |
| | Hs.82101.0 | 217996_at | 1045.52 | 278.20 | 4329.55 | 3420.52 |
| | Hs.82101.0 | 217999_s_at | 133.44 | 46.85 | 1052.79 | 719.08 |
| | Hs.82101.0 | 217997_at | 313.00 | 73.52 | 1782.89 | 1314.23 |
| SKAP2 | g4506962 | 204362_at | 55.33 | 49.87 | 5996.90 | 3468.27 |
| | Hs.52644.1 | 216899_s_at | 53.08 | 24.42 | 3468.27 | 1897.65 |
| | g4062959 | 204361_s_at | 18.13 | 13.18 | 2225.63 | 1305.15 |
| | Hs.5888.0 | 225639_at | 56.89 | 26.54 | 4904.87 | 2646.74 |

### <Quantitative RT-PCR>

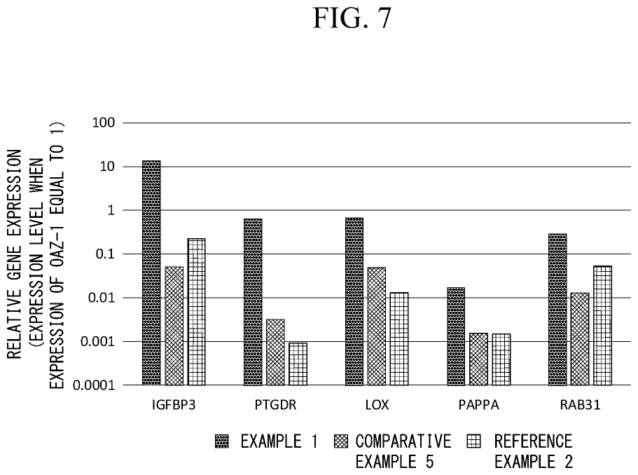

The expression levels of the IGFBP3 gene, the PTGDR gene, the LOX gene, the PAPPA gene, and the RAB31 gene were measured by the same method as that in <Quantitative RT-PCR> above for primitive gut tube cells induced to differentiate by being cultured by the methods described in Example 1, Reference Example 2 and Comparative Example 5. Information on each gene and the primer sequence thereof are shown in Table 10 (the sequences are indicated as SEQ ID NO:27 to 36 in the sequence listing). The measurement results are shown in FIG. 7.

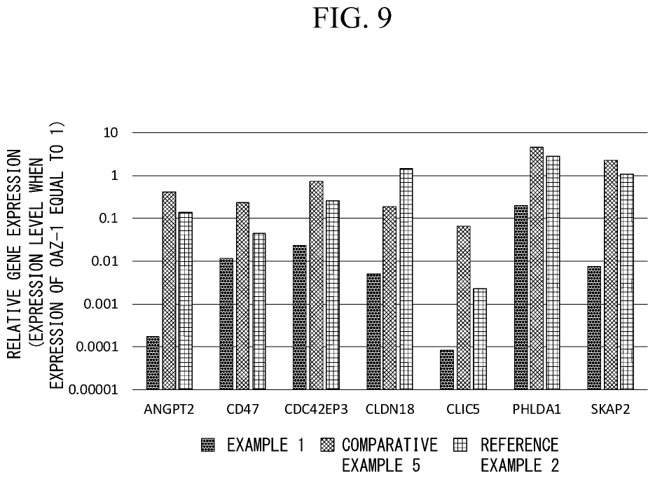

The expression levels of the ABGPT2 gene, the CD47 gene, the CDC42EP3 gene, the CLIDN18 gene, the CLIC5 gene, the PHLDA1 gene, and the SKAP2 gene were measured by the same method as that in <Quantitative RT-PCR> above for primitive gut tube cells induced to differentiate by being cultured by the methods described in Example 1, Reference Example 2 and Comparative Example 5. Information on each gene and the primer sequence thereof are shown in Table 11 (the sequences are indicated as SEQ ID NO:37 to 50 in the sequence listing). The measurement results are shown in FIG. 9. The numerical values from the graphs shown in FIG. 7 and FIG. 9 are indicated in Table 12. It was observed that, in comparison to the gene expression in the primitive gut tube cells induced to differentiate by being cultured by the method described in Example 5, the gene expression in the primitive gut tube cells induced to differentiate by being cultured by the method described in Example 1 exhibits tendencies similar to the results of the DNA microarray data.

TABLE 10

| Gene | | NCBI | | |
|---|---|---|---|---|
| Symbol | Gene name | Gene ID | Primer name | Sequence 5′→3′ |
| IGFBP3 | insulin-like growth | 3486 | IGFBP3-F | AGAATATGGTCCCTGCCGTAGA |
| | factor-binding | | IGFBP3-R | CGTCTACTTGCTCTGCATGCTG |
| | protein 3 | | | |
| PTGDR | prostaglandin D2 | 5729 | PTGDR-F | TCTTTGGGCTCTCCTCGACA |
| | receptor | | PTGDR-R | GGCAGTACTGCACGAACTTCC |
| LOX | lysyl oxidase | 4015 | LOX-F | ACCTGCTTGATGCCAACAC |
| | | | LOX-R | GTCAGATTCAGGAACCAGGTAG |
| PAPPA | pappalysin 1 | 5069 | PAPPA-F | TTTCCAGCTAGCAGTACTC |
| | | | PAPPA-R | TCCCTATGTGATGTAACTAGTC |
| RAB31 | RAB31, member | 11031 | RAB31-F | GTATTCAGACCGACTGGGTATC |
| | RAS oncogene | | RAB31-R | TAGAATACATGGCGGAAAGGTC |
| | family | | | |

TABLE 11

| | | NCBI | | |
|---|---|---|---|---|
| Gene | | Gene | | |
| Symbol | Gene name | ID | Primer name | Sequence 5′→3′ |
| ANGPT2 | angiopoietin 2 | 285 | ANGPT2-F | GCACAAAGGATGGAGACAACGA |
| | | | ANGPT2-R | GGTTGTGGCCTTGAGCGAA |
| CD47 | CD47 molecule | 961 | CD47-F | TCATCACCTTCCTCCTGTAGTC |
| | | | CD47-R | AACCTTTGCTCTCCTGTAGGT |
| CDC42EP3 | CDC42 effector | 10602 | CDC42EP3- | AGGCACTTTAGACCCATACC |
| | protein 3 | | F | |
| | | | CDC42EP3- | CCTACCTCAACAAGAAGTGTC |
| | | | R | |
| CLDN18 | claudin 18 | 51208 | CLDN18-F | CGAGCCCTGATGATCGTAG |
| | | | CLDN18-R | ATGTTGGCAAACACAGACAC |
| CLIC5 | chloride | 53405 | CLIC5-F | ACAATGATTCCCAAGGGATCAC |
| | intracellular | | CLIC5-R | CACTAAGCTGGGAGATGCATAC |
| | channel 5 | | | |
| PHLDA1 | pleckstrin | 22822 | PHLDA1-F | AGAGGGCAAGGAGATCGAC |
| | homology-like | | PHLDA1-R | GATGTGGATGCGGATACGG |
| | domain family A | | | |
| | member 1 | | | |
| SKAP2 | src kinase- | 8935 | SKAP2-F | CAATCCACTAACAAGCAGTCAAC |
| | associated | | SKAP2-R | CACCACGCTTAAATGACAACTC |
| | phosphoprotein 2 | | | |

TABLE 12
Relative expression level when expression of OAZ-1 equal to 1

| | Elevated expression | | | | |
|---|---|---|---|---|---|
| | IGFBP3 | PTGDR | LOX | PAPPA | RAB31 |
| Example 1 | 13.05428 | 0.63927 | 0.65690 | 0.01698 | 0.28091 |
| Comparative | 0.05091 | 0.00322 | 0.04744 | 0.00155 | 0.01267 |
| Example 5 | | | | | |
| Reference | 0.22029 | 0.00091 | 0.01278 | 0.00147 | 0.05338 |
| Example 2 | | | | | |

Reduced expression

| | ANGPT2 | BMPR1B-m1 | CD47 | CDC42EP3 | CLDN18 | CLIC5 | FRZB | PHLDA1 | SKAP2 |
|---|---|---|---|---|---|---|---|---|---|
| Example 1 | 0.00018 | 0.04130 | 0.01146 | 0.02352 | 0.00517 | 0.00008 | 0.08015 | 0.19997 | 0.00741 |
| Comparative | 0.40639 | 0.91840 | 0.23373 | 0.73378 | 0.18705 | 0.06539 | 4.45049 | 4.57830 | 2.30361 |
| Example 5 | | | | | | | | | |
| Reference | 0.13582 | 0.03999 | 0.04520 | 0.25191 | 1.46373 | 0.00222 | 0.05955 | 2.86514 | 1.07755 |
| Example 2 | | | | | | | | | |

1. A method for producing primitive gut tube (PGT) cells comprising a step of culturing, in the absence of a bone morphogenetic protein (BMP) signaling inhibitor, endodermal cells that have been induced to differentiate from pluripotent stem cells.

2. The method according to claim 1, wherein the step of culturing, in the absence of a bone morphogenetic protein (BMP) signaling inhibitor, endodermal cells that have been induced to differentiate from pluripotent stem cells is performed in the absence of FGF2.

3. The method according to claim 1, wherein the step of culturing, in the absence of a bone morphogenetic protein (BMP) signaling inhibitor, endodermal cells that have been induced to differentiate from pluripotent stem cells is performed in the absence of a hedgehog (HH) signaling inhibitor.

4. The method according to claim 1, wherein the step of culturing, in the absence of a bone morphogenetic protein (BMP) signaling inhibitor, endodermal cells that have been induced to differentiate from pluripotent stem cells is performed in the absence of a TGFβ signaling inhibitor.

5. The method according to claim 1, wherein the step of culturing, in the absence of a bone morphogenetic protein (BMP) signaling inhibitor, endodermal cells that have been induced to differentiate from pluripotent stem cells is a step of culturing the endodermal cells in a culture medium containing insulin, transferrin, and selenous acid.

6. The method according to claim 1, wherein the step of culturing, in the absence of a bone morphogenetic protein (BMP) signaling inhibitor, endodermal cells that have been induced to differentiate from pluripotent stem cells is a step of culturing the endodermal cells in a culture medium containing a B27 (registered trademark) supplement and/or FGF7.

7. The method according to claim 1, wherein the endodermal cells that have been induced to differentiate from pluripotent stem cells are endodermal cells that have been induced to differentiate by culturing a pluripotent stem cell population in a culture medium containing a TGFβ superfamily signaling activator, and thereafter culturing the cell population in a culture medium to which FGF2 and BMP4 are not added.

8. Primitive gut tube (PGT) cells wherein expression of at least one gene selected from the group consisting of the KIT gene, the RAP1A gene, the FGF11 gene, and the FGFR4 gene is elevated, and/or expression of at least one gene selected from the group consisting of the MDM2 gene, the CASP3 gene, and the CDK1 gene is reduced in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.

9. The primitive gut tube (PGT) cells according to claim 8, wherein expression of at least one gene selected from the group consisting of the IGFBP3 gene, the PTGDR gene, the LOX gene, the PAPPA gene, and the RAB31 gene is elevated in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.

10. The primitive gut tube (PGT) cells according to claim 8, wherein expression of at least one gene selected from the group consisting of the ANGPT2 gene, the CD47 gene, the CDC42EP3 gene, the CLDN18 gene, the CLIC5 gene, the PHLDA1 gene, and the SKAP2 gene is reduced in comparison with primitive gut tube (PGT) cells produced by culturing, in the presence of a bone morphogenetic protein (BMP) signaling inhibitor, retinoic acid or an analog thereof, a TGF-β signaling inhibitor, and a hedgehog (HH) signaling inhibitor, endodermal cells that have been induced to differentiate from pluripotent stem cells.

11. Primitive gut tube (PGT) cells wherein expression of at least one gene selected from the group consisting of the IGFBP3gene, the PTGDR gene, and the PAPPA gene is elevated, and/or expression of at least one gene selected from the group consisting of the ANGPT2 gene and the FRZB gene is reduced in comparison with endodermal cells that have been induced to differentiate from pluripotent stem cells.
